(19) United States
(12) Patent Application Publication
(10) Pub. No.: US 2007/0155835 A1
(43) Pub. Date: Jul. 5, 2007

(54) USE OF ORTHO-PHENYLPHENOL AND/OR DERIVATIVES THEREOF FOR INHIBITING THE ASEXUAL REPRODUCTION OF FUNGI

(75) Inventors: Mirko Weide, Duesseldorf (DE); Anja Schloesser, Neuss (DE); Dirk Bockmuehl, Wuppertal (DE); Roland Breves, Mettmann (DE); Andreas Bolte, Duesseldorf (DE); Thomas Gerke, Neuss (DE); Achim Schunk, Duesseldorf (DE)

Correspondence Address: HENKEL CORPORATION, THE TRIAD, SUITE 200, 2200 RENAISSANCE BLVD., GULPH MILLS, PA, 19406, US

(73) Assignee: Henkel Kommanditgesellschaft auf Aktien (Henkel KGaA), Duesseldorf (DE)

(21) Appl. No.: 11/669,468

(22) Filed: Jan. 31, 2007

Related U.S. Application Data
(63) Continuation of application No. PCT/EP05/08178, filed on Jul. 28, 2005.

(30) Foreign Application Priority Data
Aug. 5, 2004 (DE) 10 2004 038 104.6

(51) Int. Cl. A61K 31/235; A61K 31/05

(52) U.S. Cl. 514/543; 514/731

(57) ABSTRACT

The use of ortho-phenylphenol and/or derivatives thereof to inhibit the asexual reproduction of fungi, and to filter media, construction materials, construction adjuvants, textiles, furs, paper, hide, or leather. Also, washing agents, cleaning agents, rinsing agents, hand washing agents, hand dishwashing agents, automatic dishwashing agents, and agents for finishing construction materials, construction adjuvants, textiles, furs, paper, hides, or leather, that contain ortho-phenylphenol and/or derivatives thereof,

### CROSS REFERENCE TO RELATED APPLICATIONS

This application is a continuation under 35 U.S.C. §365(c) and 35 U.S.C. §120 of international application PCT/EP2005/008178, filed Jul. 28, 2005. This application also claims priority under 35 U.S.C. §119 of DE 10 2004 038 104.6, filed Aug. 5, 2004, which is incorporated herein by reference in its entirety.

### BACKGROUND OF THE INVENTION

The invention relates to the use of ortho-phenylphenol and/or derivatives thereof to inhibit the asexual reproduction of fungi, and to filter media, adhesives, construction materials, construction adjuvants, textiles, furs, paper, hide, or leather, but also washing agents, cleaning agents, rinsing agents, hand washing agents, hand dishwashing agents, automatic dishwashing agents, and agents for finishing construction materials, construction adjuvants, textiles, furs, paper, hides, or leather, that contain ortho-phenylphenol and/or derivatives thereof.

Fungi, in particular mold fungi, cause considerable problems in the field of construction biology, since the spores discharged from them into indoor air are often allergenic. Counteracting such fungi with biocidal active substances is associated with an elevated risk of creating resistance, so that after a certain time new antimicrobial substances must be found that are effective against these now-resistant microorganisms. Biocides are moreover not always environmentally and toxicologically harmless. Among the undesirable effects of the propagation of mold fungi are, in particular, discolorations (for example, on walls, joint sealing compounds, and bath surfaces) that are brought about by pigmented spores.

Sensitive textiles, for example silk or microfiber, are more and more often being processed into garments that can be washed only at 30 or 40° C. Fungi, for example the human pathogen Candida albicans, are not thereby killed. In particular following a fungal infection, fungi of this kind that adhere to garments and are not killed can cause a re-infection,

Antimicrobial substances that either inhibit the growth of fungi (fungistatics) or kill them (fungicides) have therefore previously been used. Non-selective antimicrobial substances that act against both bacteria and fungi are often used for this. A disadvantage thereof is that such biocides or biostatics used, for example, in washing and cleaning agents contaminate wastewater and thus also interfere with the functioning of the microbial treatment steps in sewage treatment plants.

It is known in the existing art that ortho-phenylphenol, used in high concentrations, is suitable for inhibiting the growth of fungi (fungistatic effect) or even for killing fungi (fungicidal effect). The existing art does not describe the fact that ortho-phenylphenol can be used to inhibit the asexual reproduction of fungi (in particular sporulation). A general inhibition of growth of course also results in an inhibition of asexual reproduction, in particular of sporulation. It is not known in the existing art that ortho-phenylphenol can diminish and/or entirely prevent the asexual reproduction of fungi, in particular sporulation, without inhibiting growth of the fungi per se.

The earlier international Patent Application PCT/EP02/14306, not previously published, describes that mono-, sesqui-, and/or diterpenes and derivatives thereof can be used to inhibit the asexual reproduction of fungi. Farnesol is cited as a particularly preferred active substance. The use of ortho-phenylphenol to inhibit the asexual reproduction of fungi is not described therein.

U.S. Pat. Nos. 4,120,970 and 3,674,510 describe that ortho-phenylphenol can be used to preserve fruits. Ortho-phenylphenol is used therein, however, at concentrations that are toxic to the fungi. What happens is therefore not that asexual reproduction itself is inhibited, as occurs according to the present invention, but that the germs are simply killed.

It is therefore the object of the invention to overcome the disadvantages of the existing art and to prevent the asexual reproduction of fungi, in particular the sporulation of mold fungi, in particular on surfaces, without killing the fungi.

Surprisingly, it has been found that the use of ortho-phenylphenol and/or derivatives thereof on or in fungus-infested materials is capable of suppressing propagation of the fungi without killing them.

The subject matter of the present invention is therefore the use of ortho-phenylphenol and/or derivatives thereof to inhibit the asexual reproduction of fungi.

According to the present invention, the term “asexual reproduction” encompasses in particular sporulation, budding, and fragmentation.

Included among the derivatives of ortho-phenylphenol are preferably esters and ethers of ortho-phenylphenol that occur by reaction with the phenolic hydroxyl group. The carboxylic acid radical of the ortho-phenylphenol ester can be in particular a C₁₋₁₈ alkylcarboxylic acid, preferably a C₁₋₁₂ carboxylic acid, or a C₆₋₁₀ aryl-C₁₋₆ alkylcarboxylic acid, such that the alkyl radical can be branched or unbranched and saturated or unsaturated. The alcohol radical of the ortho-phenylphenol ether can be, in particular, a C₁₋₁₈ alcohol, preferably a C₁₋₆ alcohol. Likewise included, according to the present invention, among the derivatives of ortho-phenylphenol are mono- or multiply substituted, in particular mono-, di-, or trisubstituted, ortho-phenylphenol, mono- or multiply substituted, in particularly mono-, di-, or trisubstituted, ortho-phenylphenol ethers and ortho-phenylphenol esters, and mono- or multiply substituted, in particular mono-, di-, or trisubstituted, biphenyl. The substituents in this context are preferably selected from the group made up of C₁₋₆ alkyl, C₁₋₆ alkoxy, C₆₋₁₀ aryl, C₆₋₁₀-aryl-C₁₋₆ alkyl, C₆₋₁₀ aryl-C₁₋₆ alkoxy, hydroxy, halogen, in particular chloro or fluoro, nitro, cyano, amino, mono-, and di-C₁₋₆ alkylamino, and benzyl. Especially to be mentioned here are ortho-phenylphenol benzoate, ortho-phenylphenol palmitate, ortho-phenylphenol cinnamate, ortho-phenylphenol acetate, and ortho-phenylphenol-O-β-D-glucopyranoside.

Further derivatives of ortho-phenylphenol that are suitable according to the present invention are esters of ortho-phenylphenol and/or of the aforementioned derivatives of ortho-phenylphenol with silicic acid, according to formulas (I) and (II). The silicic acid esters are manufactured in particular by simple transesterification of silicic acid esters (n=1) or silicic acid oligoesters (n>1) of lower alcohols with ortho-phenylphenol and/or derivatives thereof. Depending on the reaction time and conditions, the lower alcohols are cleaved and ortho-phenylphenol is bound, the alcohols along the Si—O—Si chain being exchanged more easily than the terminal alcohols

Preferred ortho-phenylphenol silicic acid esters are those according to one of formulas (I) or (II), and/or mixtures thereof. 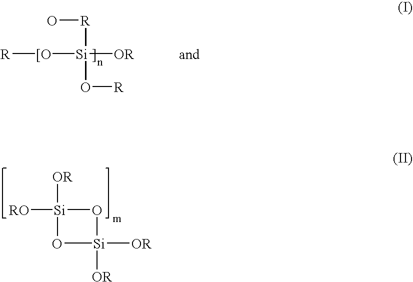
in which at least one R is ortho-phenylphenyl or a derivative thereof and all the other R, independently of one another, are selected from the group that contains H, the straight-chain or branched, saturated or unsaturated, substituted or unsubstituted C₁₋₆ hydrocarbon radicals, terpene alcohols, and polymers, and m assumes values from the range of 1 to 20 and n assumes values from the range of 1 to 100.

According to a further preferred embodiment, at least two or three of the R radicals are ortho-phenylphenyl or a derivative thereof.

The oligomerization numbers “n” of the silicic acid esters according to the present invention are between 1 and 20. In preferred compounds, n assumes values between 1 and 15, preferably between 1 and 12, and in particular between 1 and 10, with particular preference for the values 4, 5, 6, 7, and 8.

The silicic acid esters utilized according to the present invention are notable for good hydrolysis stability and can also be used in aqueous media and in manufacturing processes for granules, sealing compounds, etc. without thereby experiencing an excessive loss of activity. As a result, release of the active substance from the substances according to the present invention takes place slowly and in comparatively small quantities, so that a non-fungicidal and non-fungistatic concentration of the active substances is continuously released from the products over a longer period of time,

According to a particularly preferred embodiment, one or more polymer radicals can be present on the silicic acid esters. Those polymers that contain free hydroxyl groups are preferably used for manufacture of the silicic acid esters. The polymer radical(s) is or are selected, in particular, from starch and/or derivatives thereof cellulose and/or derivatives thereof, polyvinyl alcohol, polyols, hydroxypolydimethylsiloxanes (very particularly a α,ω-dihydroxypolydimethylsiloxane), and polyphenols, in particular polyvinyl alcohol. It is particularly preferred if a polymer radical is present on the silicic acid esters that carry ortho-phenylphenol. For utilization in sealing compounds, it is particularly preferred to use fairly short-chained polymers.

This specific embodiment has the advantage that the silicic acid esters can be individually adapted, depending on the field of application, to the particular purpose and conditions. Such polymers are particularly suitable, for example, for improving the ability of the substances to be incorporated; for enhancing adhesion, in particular to surfaces; and for influencing release properties as desired.

According to a further particular embodiment, the ortho-phenylphenol derivatives also include esters of ortho-phenylphenol, or esters of one of the aforementioned ortho-phenylphenol derivatives, with polymers. These substances, too, yield better adaptability to the purpose of an application, for example better absorption onto or adhesion to surfaces, or more favorable conditions for ability to be incorporated. Hydrolysis of this ester bond, for example upon repeated contact with water, slowly releases the active substances which can then inhibit the asexual reproduction of fungi.

Particularly preferably, such substances are produced by reaction of the ortho-phenylphenol or ortho-phenylphenol derivative with polymers of this kind that carry functional groups which are selected in particular from acid groups, acid chloride groups, ester groups, primary, secondary, and tertiary amide groups.

Polyacrylic acid, polyacrylic acid esters, polymethacrylic acid, polymethacrylic acid esters, polycarboxylic acids (in particular carboxymethyl cellulose), and copolymers of the underlying monomers (including with monomers other than those mentioned), and primary, secondary, or tertiary polyacrylamides, are preferably used according to the present invention as polymers. Chain lengths from approx. 2000 to 300,000 g/mol are particularly preferred in this context.

According to a further preferred embodiment, the ortho-phenylphenol derivative is manufactured by reacting ortho-phenylphenol and/or one of the aforementioned ortho-phenylphenol derivatives with monomers or polymers that carry one or more isocyanate groups. The urethanes resulting from reaction of an alcohol function with an isocyanate group likewise hydrolyze slowly and release the active substance in controlled fashion.

The use of monomeric aliphatic or aromatic mono-, di-, and/or triisocyanates is preferred. The resulting urethanes or (when isocyanates having multiple isocyanate groups are utilized) polyurethanes can likewise hydrolyze and release the active substances slowly.

Preferred as monoisocyanates are, for example, the linear or branched aliphatic monoisocyanates having 6 to 44 C atoms, for example hexyl isocyanate, heptyl isocyanate, octyl isocyanate, nonyl isocyanate, decyl isocyanate, undecyl isocyanate, dodecyl isocyanate, tridecyl isocyanate, quaterdecyl isocyanate, pentadecyl isocyanate, hexadecyl isocyanate, heptadecyl isocyanate, octadecyl isocyanate, and the corresponding higher homologs of this series. Also preferred are aromatic monoisocyanates such as phenyl isocyanate, benzyl isocyanate, or biphenyl isocyanate.

Particularly preferred as diisocyanates (Q(NCO)₂) are those in which o is selected from an aliphatic, optionally substituted hydrocarbon radical having 4 to approximately 15 carbon atoms, an aromatic, optionally substituted hydrocarbon radical having 6 to approximately 15 carbon atoms, or an optionally substituted araliphatic hydrocarbon radical having 7 to approximately 15 carbon atoms. To be mentioned here, for example, are tetramethylene diisocyanate, hexamethylene diisocyanate, dodecamethylene diisocyanate, dimer fatty acid diisocyanate, 1,4-diisocyanatocyclohexane, 1-isocyanato-3,3,5-trimethyl-5-isocyanatomethylcyclohexane (IDPI), 4,4′-diisocyanatodicyclohexylmethyl, 4,4′-diisocyanatodicyclohexylpropane-2,2, 1,3- and 1,4-diisocyanatobenzene, 2,4- or 2,6-diisocyanatotoluene or mixtures thereof, 2,2′-, 2,4 or 4,4′-diisocyanatodiphenylmethane, tetramethylxylylene diisocyanate, p-xylylene diisocyanate, and mixtures made up of these compounds.

Toluene diisocyanate, hexamethylene diisocyanate, and meta-tetramethylxylylene diisocyanate are particularly preferred,

Especially appropriate as triisocyanates are aromatic triisocyanates such as, for example, thiophosphoric acid tris-(p-isocyanato)phenyl ester, triphenylmethane-4,4′,4″-triisocyanate, and in particular the various isomeric trifunctional homologs of diphenylmethane diisocyanate (MDI).

Also suitable as triisocyanates are adducts of diisocyanates and low-molecular-weight triols, in particular the adducts of aromatic diisocyanates and triols such as, for example trimethylolpropane or glycerol. The aforementioned limitations with respect to the diisocyanate content, as well as the content of polyisocyanates having a functionality >3, also apply to these adducts.

Aliphatic triisocyanates such as, for example, the biuretization product of hexamethylene diisocyanate (HDI) or the isocyanidation product of HDI, or also the same trimerization products of isophorone diisocyanate (IPDI), are also suitable for the compositions according to the present invention.

Polyisocyanates are the dimerization or trimerization products of the diisocyanates already mentioned as preferred. Examples of suitable isocyanates are the dimerization or trimerization products of the diisocyanates 2,4-toluylene diisocyanate (2,4-TDI). 2,6-toluylene diisocyanate (2,6-TDI), or a mixture of the aforesaid isomers, 2,2′-diphenylmethane diisocyanate (2,2-MDI). 2,4′-diphenylmethane diisocyanate (2,4′-MDI), 4,4′-diphenylmethane diisocyanate (4,4′-MDI), 1,5-naphthylene diisocyanate (NDI), 1,4-phenylene diisocyanate, 1,3-tetramethylxylylene diisocyanate (TMXDI), hydrogenated MDI (HMDI), isophorone diisocyanate (IPDI), hexamethylene diisocyanate-1,6 (HDI) 2-isocyanatopropylcyclohexyl isocyanate (IPCI), 2-butyl-2-ethylpentamethylene diisocyanate (BEPDI), lysine diisocyanate (LDI), 1,12-dodecyl diisocyanate, cyclohexyl 1,3- or 1,4-diisocyanate, 2-methylpentamethylene diisocyanate (MPDI) or the like, for example containing urethane, allophanate, urea, biuret, uretidone, carbidiimide, or ketoneimine groups, such as those resulting from dimerization or trimerization of the aforementioned diisocyanates. Particularly suitable are compounds carrying oligomeric or polymeric isocyanate groups, such as those that, for example, occur in isocyanate manufacture or remain in the distillation sump as residual products upon distillation of crude isocyanate products. Examples of materials that are particularly suitable in this connection are crude MDI as obtainable directly after the manufacture of MDI, and polymeric MDI that remains in the distillation sump after the distillation of MDI from crude MDI.

It is preferred to add a corresponding quantity of ortho-phenylphenol or ortho-phenylphenol derivative to the monomers, and thereby to generate corresponding monomers. For example, substances can be produced that, depending on the monomers used (monoisocyanates, diisocyanates, triisocyanates, or polyisocyanates), carry one or more, in particular one, two, or three, ortho-phenylphenol radicals. It is also possible to produce, via a polymerization reaction, a polymer chain having terminal ortho-phenylphenol radicals.

Such monomers or polymers can be used in sealing compounds, for example directly in the cartridge or in a separate compartment as an additive. The corresponding ortho-phenylphenol derivatives can also, in the production of the sealing compounds (especially those based on urethane) be added directly to the monomers of the sealing compounds. The use of reaction products of mono-, di-, and/or triisocyanates having ortho-phenylphenols or derivates thereof in sealing compounds in particular preferred.

Suitable chain-extending agents that can additionally be used in the context of a polymerization reaction for manufacture of the substances to be used according to the present invention are, for example, polyvalent alcohols such as ethylene glycol, propylene glycol, propanediol-1,3, butanediol-1,4, hexanediol-1,6, trimethylolpropane, glycerol, pentaerythritol, sorbitol, mannitol, or glucose. Low-molecular-weight polyesterdiols such as succinic acid, glutaric acid, or adipic acid bis-(hydroxyethyl)esters, or a mixture of two or more thereof, or low-molecular-weight diols having ether groups, such as diethylene glycol, triethylene glycol, tetraethylene glycol, dipropylene glycol, tripropylene glycol, or tetrapropylene glycol can concurrently be used. Also suitable are amines such as ethylenediamine, hexamethylenediamine, piperazine, 2,5-dimethylpiperazine, 1-amino-3-aminomethyl-3,5,-trimethylcyclohexane (isophoronediamine, IPDA), 4,4′-diaminodicyclohexylmethane, 1,4-diaminocyclohexane, 1,2-diaminopropane, hydrazine, hydrazine hydrate, amino acid hydrazides such as 2-aminoacetic acid hydrazide, or bishydrazides such as succinic acid bishydrazide. The concurrent use of small quantities of compounds that are tri- or higher-functional in the context of an isocyanate polyaddition reaction is likewise possible in order to achieve a certain degree of branching, as is the possible concurrent use (already mentioned) of tri- or higher-functional polyisocyanates for the same purpose. Univalent alcohols such as n-butanol or n-dodecanol and stearyl alcohol can be concurrently used in small quantities.

According to a further preferred embodiment, carrier-bound forms of the ortho-phenylphenol and/or of the aforementioned ortho-phenylphenol derivatives can also be used according to the present invention, in particular clathrate molecules that are charged with ortho-phenylphenol and/or with a derivative thereof.

“Clathrate molecules” are to be understood in the context of the present invention as, in particular, those organic macrocyclic molecules that have a cage-like three-dimensional structure and are capable, as so-called host molecules, of enclosing one or more so-called guest molecules. Preferably only one guest molecule is enclosed in each case.

The intended slow release of the compounds suitable for inhibition of the asexual reproduction of fungi can also take place by equilibration from an (often not covalent) bonds or by complexing of the compound from a clathrate molecule.

Because of the fairly hydrophobic outer shell of the clathrate substances, the charged clathrate molecules are particularly easy to incorporate into the products according to the present invention, in particular into those of a more hydrophobic nature.

A particularly great advantage of the use of clathrate molecules is the fact that it is possible after an extended period of time, by recharging the clathrate molecules, to replace in the products the substances that have diffused out. Concentrated solutions of the aforesaid active substances are especially suitable for this. In this connection it is likewise possible to manufacture products that do not a priori contain the free active substances in complexed or bound fashion in the clathrate molecules, but instead are charged with them only in an application situation. In terms of formulation technology, this is useful for areas of use known to one skilled in the art.

Cucurbiturils, calixarenes, calixresorcarenes, cyclodextrins, cyclophanes, crown ethers, fullernes, cryptophanes, carcerands, hemicarcerands, cyclotriveratrylenes, spherands, and cryptands may be mentioned as organic clathrate molecules.

The cucurbiturils, calixarenes, and calixresorcarenes are particularly preferred according to the present invention, very particularly cucurbiturils.

Cucurbiturils and their manufacture are described in the literature, for example in WO 00/68332 and EP-A 1 094 065 and the further literature cited therein. A cucurbituril usable for purposes of the invention is to be understood essentially as any substance that is described in the literature as belonging to this class of compounds. Included herein by definition are the cucurbiturils and substituted cucurbiturils described in WO 00/68232, and the cucurbituril derivatives described in EP-A 1 094 065. In place of a homogeneous cucurbituril, substituted cucurbituril, or cucurbituril derivative, mixtures of two or more such compounds can also be used. When a cucurbituril is referred to hereinafter, and unless expressly indicated otherwise, it is similarly to be understood as a chemically homogeneous cucurbituril or also a mixture of two or more cucurbiturils, substituted cucurbiturils, and/or cucurbituril derivatives. Quantitative indications of cucurbiturils correspondingly, unless expressly indicated otherwise, always refer to the total quantity of the one or more cucurbiturils, substituted cucurbiturils, and/or cucurbituril derivatives.

Preferred for purposes of the present invention are cucurbit[n]urils having a ring size of 5 to 11 as well as mixtures thereof, cucurbit[6]uril as well as mixtures having a predominant proportion of cucurbit[6]uril being particularly preferred.

Calix[n]arenes according to formula (III) can additionally be used. 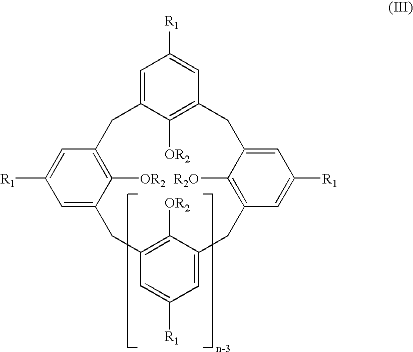
in which

R₁ is selected from R₁═H, alkyl, aryl, alkenyl, alkinyl as well as substituted alkyls, aryls, alkenyls, alkinyls which carry groups that are selected from —OH, —OR′, —NH₂, —NHR′, —NR′R″, NR′R″R′″⁺, NO₂, halogen, SO₃H, SO₃M (M=alkali metals, alkaline-earth metals), carboxylic acids, ketones, aldehydes, amides, esters, —SO₂NH₂, —SO₂NHR, —SO₂NR′R″, —SO₂-halogen, sulfur-, phosphorus-, silicon-containing groups,

and

R₂ is selected from R₂═H, alkyl, aryl, alkenyl, alkinyl as well as substituted alkyls, aryls, alkenyls, alkinyls which carry groups that are selected from —OH, —OR′, —NH₂, —NHR′, —NR′R″, —NR′R″R′″⁺, —NO₂, halogen, —SO₃H, —SO₃M (M=alkali metals, alkaline-earth metals), carboxylic acids, ketones, aldehydes, amides, esters, —SO₂NH₂, —SO₂NHR′, —SO₂NR′R″, —SO₂-halogen, sulfur-, phosphorus-, silicon-containing groups,

R′, R″, R′″ being selected, independently of one another, from H, alkyl, aryl, alkenyl, alkinyl, substituted alkyls, aryls, alkenyls, alkinyls.

Calixarenes according to formula (III) for which

R₁ is selected from R₁═H, alkyl aryl, alkenyl alkinyl as well as substituted alkyls, aryls, alkenyls, alkinyls which carry groups that are selected from —OH, —OR′, —NH₂, —NHR′, —NR′R″, NR′R″R′″⁺, NO₂, halogen, SO₃H, carboxylic acids, ketones, aldehydes, amides, esters, —SO₂NR′R″,

and

R₂ is selected from R₂═H, alkyl, aryl, alkenyl, alkinyl as well as substituted alkyls, aryls, alkenyls, alkinyls which carry groups that are selected from —OH, —OR′, —NH₂, —NR′R″, —NR′R″R′″⁺, —NO₂, halogen, —SO₃H, carboxylic acids, ketones, aldehydes, amides, esters, —SO₂NR′R″,

R′, R″, R′″ being selected, independently of one another, from H, alkyl, aryl, alkenyl, alkinyl as well as substituted alkyls, aryls, alkenyls, alkinyls, are preferred.

Calix[n]arenes having a ring size n=4 to 12, as well as mixtures thereof, are preferred for purposes of the present invention, calix[6]- and/or calix[4]arenes, as well as mixtures having a predominant proportion of calix6]- and/or calix[4]arenes, being particularly preferred.

Additionally to be used are calix[n]resorcarenes, also known as resorcinarenes, according to formula (IV), in which n indicates the number of chain members and can be 4 or 6: 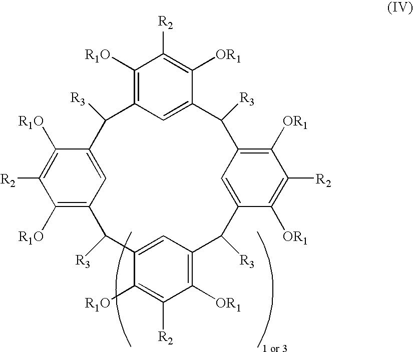
in which R₁, R₂, and R₃ are selected from:

R₁═H, alkyl, aryl, alkenyl, alkinyl as well as substituted alkyls, aryls, alkenyls, alkinyls which carry groups that are selected from —OH, —OR, —NH₂, —NHR′, —NR′R″, NR′R″R′″⁺, —NO₂, halogen, SO₃H, SO₃M (M=alkali metals, alkaline-earth metals), carboxylic acids, ketones, aldehydes, amides, esters, SO₂NH₂, SO₂NHR, SO₂NR₂, SO₂-halogen, sulfur-, phosphorus-, silicon-containing groups,

and

R₂, R₃ are selected, independently of one another, from R₂, R₃═H, alkyl, aryl, alkenyl, alkinyl as well as substituted alkyls, aryls, alkenyls, alkinyls which carry groups that are selected from —OH, —OR, —NH₂, —NHR, —NR′R″, NR′R″R′″⁺, —NO₂, halogen, —SO₃H, —SO₃M (M=alkali metals, alkaline-earth metals), carboxylic acids, ketones, aldehydes, amides, esters, —SO₂NH₂, —SO₂NHR, —SO₂NR₂, —SO₂-halogen, sulfur-, phosphorus-, silicon-containing groups,

and in which R′, R″, R′″ are selected, independently of one another, from H, alkyl, aryl, alkenyl, alkinyl as well as substituted alkyls, aryls, alkenyls, alkinyls.

Calix[4]resorcarene and/or calix[r]resorcarenes according to formula (IV) for which

R₁ is selected from R₁═H, alkyl, aryl, alkenyl, alkinyl as well as substituted alkyls, aryls, alkenyls, alkinyls which carry groups that are selected from —OH, —OR′, —NH₂, —NHR′, —NR′R″, NR′R″R′″⁺, NO₂, halogen, SO₃H, carboxylic acids, ketones, aldehydes, amides, esters, —SO₂NR′R″,

and

R₂, R₃ are selected, independently of one another, from R2, R₃═H, alkyl, aryl, alkenyl, alkinyl as well as substituted alkyls, aryls, alkenyls, alkinyls which carry groups that are selected from —OH, —OR′, —NH₂, —NR′R″, —NR′R″R′″⁺, —NO₂, halogen, —SO₃H, carboxylic acids, ketones, aldehydes, amides, esters, —SO₂NR′R″,

R′, R″, R′″ being selected, independently of one another, from H, alkyl, aryl, alkenyl, alkinyl as well as substituted alkyls, aryls, alkenyls, alkinyls, are preferred.

It is particularly preferred if R₂═R₃, i.e. R₂ and R₃ represent the same substituents.

According to a further embodiment, the carrier-bound forms of the ortho-phenylphenol or ortho-phenylphenol derivative are contained in a quantity of up to 50 wt %, preferably a quantity from 1 to 20 wt %, and in particular a quantity from 5 to 15 wt %.

Advantageously, the fungi are neither inhibited in their growvth nor killed in the context of the utilization according to the present invention, but asexual reproduction is merely inhibited or suppressed. Selective pressure for the creation of resistance is therefore low.

It has been found, surprisingly, that the use of ortho-phenylphenol and/or derivatives thereof can inhibit the asexual reproduction of fungi better, i.e. at a lower concentration, than farnesol.

A further advantage of the invention is that ortho-phenylphenol and/or derivatives thereof are already effective in low final concentrations as compared with fungicides or fungistatics, and therefore need cause almost no concern for undesired side effects.

According to a preferred embodiment of the present invention, ortho-phenylphenol and/or derivatives thereof are used to inhibit sporulation. “Sporulation” is to be understood as the formation of both reproductive forms, e.g. conidia, gonitocytes, sporangiospores, arthrospores, blastospores, and organs associated therewith (e.g. conidiophores) and permanent forms (e.g. chlamydospores).

Because mold fungus spores are ubiquitously present in indoor air, a mold infestation cannot in principle be prevented. Inhibition of sporulation of the growing fungal colonies, however, offers the possibility of considerably reducing the risk of a mold fungus allergy, and completely halting or considerably slowing propagation of the fungus. Discolorations resulting from sporulation are likewise greatly reduced or completely prevented.

The use of ortho-phenylphenol and/or derivatives thereof to inhibit sporulation offers the further advantage that the concentration required for use in order to inhibit sporulation is, surprisingly, once again definitely lower as compared with other sesquiterpenes, for example farnesol. A comparable effect can thus be achieved even with a lower concentration of active substance.

According to a preferred embodiment, ortho-phenylphenol and/or derivatives thereof are used in final concentrations such that they do not have a fungicidal (fungus-killing) or fungistatic (fungus growth-inhibiting) effect. A particular advantage of this embodiment is that the risk of creating resistance is low as compared with the substances being utilized, since the fungi are neither killed nor is their growth inhibited. These minimum inhibition concentrations can easily be determined in a manner known to one skilled in the art.

In a further particular embodiment, ortho-phenylphenol and/or a derivative thereof are used, optionally in carrier-bound form, in combination with a biocidal, in particular fungicidal agent, very low, in particular non-fungicidal and non-fungistatic, concentrations of ortho-phenylphenol being utilized. This combined utilization can advantageously yield a synergistic effect, in that on the one hand germs are killed, or their maturation is suppressed, by the biocide, and on the other hand any surviving germs have their asexual reproduction inhibited by the ortho-phenylphenol and/or derivative thereof. In this application form as well, the creation of resistance to ortho-phenylphenol is prevented because of the low concentration of ortho-phenylphenol. Agents described according to the present invention that contain, in addition to ortho-phenylphenol and/or a derivative thereof, at least one biocide, in particular fungicide, are therefore also a subject of the present invention. The biocide can be selected, in particular, from biocides such as those described in the following citations.

K. H. Wallhäuser, “Praxis der Sterilisation, Desinfektion, Konservierung” [Sterilization, disinfection, preservation practice], 5th revised ed., 1995 (ISBN 3134163055), Georg Thieme Verlag, Stuttgart, in particular Table 1.60 (pp. 103-105), Table 5.11 (p. 406), Table 5.12 (p. 407), Table 5.13, Table 5.14, Table 5.15 (pp. 412414), Table 6.5, Table 6.6 (p. 426), Tables 6.7 and 6.8, page 434, FIGS. 6.4, 6.5, 6.6, and 6.8, Table 6.13 (p. 436), and Chapter 7 (“Antimikrobielle Wirkstoffe” [Antimicrobial active substances], pp. 465 ff.),

S. S. Block, “Disinfection, Sterilization, and Preservation”, 5th edition, 2000 (ISBN 0-683-30740-1), Lippincott Williams & Wilkens, Philadelphia, in particular Tables 7.2, 8.1, 8.2, 9.5, 9.6, 9.7. 9.11, 9.12, 9.13, 9.14, 10.3, 12.2-12.6, 13.1-134, 14.1, 14.2, 14.6, 14.7, 14.8, 14.17, 14.19-14.25, 15.1, 16.2. 17.2, 17.3,18.1, 18.2, 19.1, and 20.2, and FIGS. 13.1 and 20.1-20.4,

List of disinfection methods tested according to the “Richtlinien für die Prüfung chemischer Desinfektionsmittel” [Guidelines for testing chemical disinfectants] and found by the Deutsche Gesellschaft für Hygiene und Mikrobiologie [German hygiene and microbiology society] to be effective, mhp-Verlag GmbH, Wiesbaden.

Ortho-phenylphenol and/or derivatives thereof are contained in the agents according to the present invention preferably in quantities from 0.000001 to 2 wt %. A particular advantage of this embodiment is that only low concentrations of these substances need to be present in order to decrease or substantially entirely prevent asexual propagation of the fungi. Preferably ortho-phenylphenol and/or derivatives thereof are contained at 0.00001 to 1 wt %, and in particular 0.00001 to 0.1 wt %. Ranges between 0.00005 and 0.05 wt %, especially between 0.00005 and 0.005 wt %, are particularly preferred.

The concentrations that lead to the desired result in the final product are significantly lower than those indicated, since dilutions must be taken into account for many products, For washing agents, for example, a dilution factor (washing-agent concentrate:water ratio) of 1:20 to 1:200 must be considered. The dilution ratio for washing agents is often between 1:60 and 1:100, for example 1:80. In the completed application solution, concentrations in particular from 0.001 to 1 wt % exhibit a particularly good sporulation-inhibiting effect. Preferably 0.001 to 0.1 wt %, for example 0.001 wt %, is used.

The action according to the present invention of ortho-phenylphenol and/or derivatives thereof is suitable in particular for inhibiting asexual reproduction of al fungi that are included in the strain lists “DSMZ—List of Filamentous Fungi” and “DSMZ—List of Yeasts” of the DSMZ (Deutsche Stammsammlung von Mikroorganismen und Zellkulturen GmbH [German microorganism and cell culture strain collection], Braunschweig). The lists may be viewed on the Internet at the respective addresses ( ) and (http://www.dsmz.de/species/yeasts.htm).

The substances used according to the present invention, ortho-phenylphenol and/or derivatives thereof, are preferably used to inhibit the asexual reproduction of fungi. Included thereamong are, for example, the human-pathogenic species of the classes Ascomycota, Basidiomycota, Deuteromycota, and Zygomycota, in particular all species of the genera Aspergillus, Penicillium, Cladosporium, and Mucor, the human-pathogenic forms of Candida, and Stachybotrys, Phoma, Alternaria, Aureobasidium, Ulocladium, Epicoccum, Stemphyllium, Paecilomyces, Trichoderma, Scopulariopsis, Wallernia, Botrytis, Verticillium, and Chaetonium.

The Ascomycota here encompass, in particular, all species of the genera Aspergillus, Penicillium und Cladosporium. These fungi form spores that, upon contact with the skin or the respiratory tract, have a great potential for triggering allergy. Included among the Basidiomycota, for example. Is Cryptococcus neoformans. Included among the Deuteromycota are all the genera known as mold fungi, in particular those that, because of the absence of a sexual phase, are not assigned to the classes Ascomycota, Basidiomycota, or Zygomycota.

The utilization according to the present invention of ortho-phenylphenol and/or derivatives thereof is particularly preferred for inhibiting sporulation in all species of the genus Aspergillus, very particularly preferred in species that are selected from Aspergillus aculeatus, Aspergillus albus, Aspergillus alliaceus, Aspergillus asperescens, Aspergillus awamori, Aspergillus candidus, Aspergillus carbonarius, Aspergillus carneus, Aspergillus chevalieri, Aspergillus chevalieri var. intermedius, Aspergillus clavatus, Aspergillus ficuum, Aspergillus flavipes, Aspergillus flavus, Aspergillus foetidus, Aspergillus fumigatus, Aspergillus giganteus, Aspergillus humicola, Aspergillus intermedius, Aspergillus japonicus, Aspergillus nidulans, Aspergillus niger, Aspergillus niveus, Aspergillus ochraceus, Aspergillus oryzae, Aspergillus ostianus, Aspergillus parasiticus, Aspergillus parasiticus var. globosus, Aspergillus penicillioides, Aspergillus phoenicis, Aspergillus rugulosus, Aspergillus sclerotiorum, Aspergillus sojae var. gymnosardae, Aspergillus sydowi, Aspergillus tamarii, Aspergillus terreus, Aspergillus terricola, Aspergillus toxicarius, Aspergillus unguis, Aspergillus ustus, Aspergillus versicolor, Aspergillus vitricolae and Aspergillus wentii.

According to a particularly preferred embodiment, ortho-phenylphenol and/or derivatives thereof are used in very particularly preferred fashion to inhibit sporulation in species of the genus Aspergillus that are selected from Aspergillus flavus and Aspergillus nidulans.

Further subjects of the present invention are washing agents, cleaning agents, rinsing agents, hand washing agents, hand dishwashing agents, automatic dishwashing agents, and agents for finishing filter media, construction materials, construction adjuvants, textiles, furs, paper, hides, or leather that contain ortho-phenylphenol and/or derivatives thereof for the inhibition of asexual reproduction.

Further subjects of the present invention are filter media, construction materials, construction adjuvants, textiles, furs, paper, hides, or leather that contain ortho-phenylphenol and/or derivatives thereof and/or have been finished with an agent according to the present invention,

Finishing of the paper, textiles, wall hanging materials, furs, hides, or leather is accomplished in the manner known to one skilled in the art, for example by immersion of the paper or the textiles, furs, hides, or leather into a suitably concentrated solution of an agent according to the present invention.

Finishing of the filter media, construction materials, or construction adjuvants is accomplished, for example, by mechanical incorporation or application of a suitably concentrated solution of an agent according to the present invention into or onto the filter media, construction materials, or construction adjuvants. Ortho-phenylphenol and solutions of ortho-phenylphenol, preferably in organic solvents, can advantageously be applied or incorporated particularly effectively onto or into such construction materials and adjuvants. Supplementary finishing of the construction materials or adjuvants, or recharging after extended use of previously finished construction materials or adjuvants, for example in the case of sealing compounds, by application of the agents according to the present invention, is therefore easily possible.

The construction materials or construction adjuvants finished according to the present invention are preferably selected from adhesive, sealing, filling, and coating compounds, plastics, lacquers, paints, wall plaster, mortar, paving plaster, concrete, insulating materials, and primers. Particularly preferred construction materials or construction adjuvants are joint sealing compounds (e.g. silicone-containing joint sealing compounds), wallpaper paste, wall plaster, carpet retention agents, silicone adhesives, dispersion paints, indoor and/or outdoor coating compounds, and tile adhesives.

Sealing compounds, and in particular joint sealing compounds, typically contain organic polymers and, in many cases, mineral or organic fillers and other additives.

Suitable polymers are, for example, thermoplastic elastomers such as those described in the Applicant's DE-A-3602526, preferably polyurethanes and acrylates. Suitable polymers are also recited in the Applicant's German Applications DE-A-3726547, DE-A-4029504, and DE-A-4009095 and in DE-A-19704553 and DE-A-4233077, to the entirety of which reference is hereby made.

The sealing compounds (sealants or sealant mixtures) according to the present invention preferably contain 0.000001 to 2, in particular 0.00001 to 1, particularly preferably 0.00001 to O.1, especially 0.00005 to 0.05, in particular 0.00005 to 0.005 wt % ortho-phenylphenol and/or derivatives thereof,

According to the present invention, finishing of the sealants according to the present invention can be accomplished both in the cured state or in the state cured below 60° C. Sealants are, in the context of the present invention, materials in accordance with DIN EN 26927, in particular those that cure plastically or elastically as sealants. The sealants according to the present invention can contain all additives typical of the corresponding sealing compounds, such as e.g. typical thickeners, reinforcing fillers, crosslinkers, crosslinking catalysts, pigments, adhesion agents, or other volume extenders. Ortho-phenylphenol and/or derivatives thereof can be incorporated by being dispersed in in a manner known to one skilled in the art, e.g. by the use of dispersion devices, kneaders, planetary mixers, etc., with moisture and oxygen excluded, both into the completed sealing compounds and into portions thereof or together with one or more components of the sealing compounds.

The treatment of previously cured, crosslinked sealing-compound surfaces can itself be performed by the application of solutions or suspensions of the substance used according to the present invention, by the fact that the active substance is transported into the sealing compound by swelling or diffusion.

Sealants usable according to the present invention can be manufactured on a silicone, urethane, and acrylic basis, or e.g. on an MS-polymer basis. Urethane-based sealants are disclosed, for example, in Ullmann's Encyclopedia of Industrial Chemistry (8th ed. 2003, Chapter 4), and in U.S. Pat. No. 4,417,042. Silicone sealants are known to one skilled in the art, for example from EP 0 118 030 A, EP 0 316 591 A, EP 0 327 847 A, EP 0 553 143 A, DE 195 49 425 A, and U.S. Pat. No. 4,417,042. Examples of acrylic sealants are disclosed in WO 01/09249 or U.S. Pat. No. 5,077,360, among others. Examples of sealants based on MS polymers are disclosed, for example, in EP 0 824 574, U.S. Pat. No. 3,971,751, U.S. Pat. No. 4,960,844, U.S. Pat. No. 3,979,344, U.S. Pat. No. 3,632,557, DE 4029504, EP 601 021, or EP 370 464.

Systems that crosslink at room temperature, as described e.g. in EP 0 327 847 or U.S. Pat. No. 5,077,360, are particularly preferred. These can be single- or multi-component systems—in the multi-component systems, the catalyst and crosslinker can be present separately (disclosed e.g. in U.S. Pat. Nos. 4,891,400 and 5,502,144)—or other so-called silicone RTV 2K systems, in particular platinum-free systems.

Particularly preferred are so-called single-component systems, which contain all the ingredients for constituting a sealing compound, are stored out of contact with atmospheric moisture and/or atmospheric oxygen, and cure at the location of use by reacting with atmospheric oxygen. The so-called neutral silicone systems, in which the reaction of crosslinkers with the water in ambient air does not result in corrosive, acid, basic, or odor-intensive cleavage products, are particularly preferred. Examples of such systems are disclosed in DE 195 49 425, U.S. Pat. No. 4,417,042, or EP 0 327 847.

The sealing compounds and, in particular, joint sealing compounds can contain aqueous or organic solvents. Suitable organic solvents are hydrocarbons such as cyclohexane, toluene, or also xylene or petroleum ether. Further solvents are ketones such as methylbutyl ketone, or chlorinated hydrocarbons.

The sealing compounds can additionally contain further rubber-like polymers. Relatively low-molecular-weight commercially available grades of polyisobutylene, polyisoprene, or also polybutadiene/styrene are appropriate here. The concurrent use of degraded natural rubber or neoprene rubber is also possible. It is also possible to use here grades that remain flowable at room temperature, which are often referred to as “liquid rubber.”

The sealing compounds according to the present invention can be used to seal or join to one another a very wide range of materials. Utilization on concrete, glass, plaster, and/or enameled surfaces, and on ceramic and porcelain, may chiefly be considered here. Also possible, however, is the joining or sealing of shaped parts or profiles made of aluminum, steel, zinc, or even plastics such as PVC or polyurethane or acrylic resins. Lastly, the sealing of wood or wood materials to a wide variety of other materials may be mentioned.

The durability of joint sealing compounds is usually achieved by adding finely particulate solids, also called fillers. These can be divided into those of an organic nature and those of an inorganic nature. Silicic acid/silicon dioxide (coated or uncoated), chalk (coated or uncoated), and/or zeolites can be preferred as inorganic fillers Zeolites can furthermore also function as drying agents. A suitable organic filler is, for example, PVC powder. The fillers generally make a substantial contribution to the fact that the sealing compound possesses a requisite internal cohesion after use, thus preventing runoff or bulging of the sealing compound out of vertical joints. The aforesaid additives or fillers can be divided into pigments and thixotroping fillers, also referred to as thixotroping agents.

Suitable thixotroping agents are the known thixotroping agents such as bentone, kaolins, or also organic compounds such as hydrogenated castor oil or derivatives thereof with multifunctional amines, or the reaction products of stearic acid or ricinoleic acid with ethylenediamine. The concurrent use of silicic acid, in particular silicic acid from pyrolysis, has proven particularly favorable. Swellable polymer powders are furthermore essentially suitable as thixotroping agents. Examples thereof are polyacrylonitrile, polyurethane, polyvinyl chloride, polyacrylic acid esters, polyvinyl alcohols, polyvinyl acetates, and the corresponding copolymers. Particularly good results can be obtained with finely particulate polyvinyl chloride powder As well as the thixotroping agents, adhesion promoters such as, for example, mercaptoalkylsilane can additionally be used. It has proven useful in this context to use a monomercaptoalkyltrialkoxysilane. Mercaptopropyltrimethoxysilane, for example, is commercially available.

The properties of a joint sealing compound can be even further improved if further components are added to the plastic powder utilized as a thixotroping agent. These are substances that fall into the category of plasticizers, or swelling agents and swelling adjuvants, used for plastics.

Possibilities, in particular for urethane- or acrylic-based sealing compounds, are e.g. plasticizers from the class of the phthalic acid esters. Examples of usable compounds from this substance class are dioctyl phthalate, dibutyl phthalate, and benzyl butyl phthalate Further suitable substance classes are chlorinated paraffins, alkylsulfonic acid esters of e.g., phenols or cresols, and fatty acid esters.

Silicone oils, particularly preferably polydimethylsiloxanes, as well as hydrocarbons and/or mixtures thereof, of which in particular hydrocarbons or mixtures thereof having a boiling point higher than 200° C., in particular higher than 230° C., are well suited as plasticizers for the silicone sealing compounds.

Those low-molecular-weight organic substances that are miscible with the polymer powder and the plasticizer are usable as swelling agents. Swelling agents of this kind may be inferred from the relevant plastics and polymer manuals for those skilled in the art. Esters, ketones, aliphatic hydrocarbons, aromatic hydrocarbons, and aromatic hydrocarbons with alkyl substituents serve as preferred swelling adjuvants for polyvinyl chloride powder.

The pigments and dyes used are the substances known for these applications, such as titanium dioxide, iron oxides, and carbon black.

In order to improve shelf stability, it is known to add to the sealing compounds stabilizers such as benzoyl chloride, acetyl chloride, toluenesulfonic acid methyl ester, carbodiimides, and/or polycarbodiimides. Olefins having 8 to 20 carbon atoms have proven to be particularly good stabilizers. In addition to the stabilizing effect, these can also perform the functions of plasticizers or swelling agents. Olefins having 8 to 18 carbon atoms are preferred, particularly if the double bond is located in the 1,2-position. The best results are obtained when the molecular structure of these stabilizers is linear.

With the use according to the present invention of ortho-phenylphenol and/or derivatives thereof to inhibit the asexual reproduction of fungi, the problem of creating resistance as a result of biocidal active ingredients is circumvented. When used in mold-susceptible construction materials and adjuvants, in particular in adhesives, coating compounds, and sealing compounds, particularly preferably in joint sealing compounds, several desirable effects are achieved because of the inhibition of sporulation:

a) prevention of discoloration due to pigmented spores;

b) delayed propagation of the mold infestation;

c) decreased allergy impact.

A further preferred subject of the present invention is wallpaper adhesives containing 0.000001 to 2 wt % ortho-phenylphenol or derivatives thereof. Examples are wallpaper pastes made from aqueous solutions of hydrocolloids such as methyl cellulose, methylhydroxypropyl cellulose, or water-soluble starch derivatives. Aqueous dispersions of film-forming high-molecular-weight substances such as polyvinyl acetate can also be used, especially in combination with the aforementioned cellulose and starch derivatives.

Regarding filter media, all known types can be used provided they are suitable for use in water- or air-filtration systems. Particularly to be mentioned are filter materials made of cellulose, glass fibers, PVC fibers, polyester fibers, polyamide fibers, in particular nylon fibers, nonwoven fibers, sintered materials, and membrane filters.

The concentration in the agents according to the present invention of ortho-phenylphenol and/or derivatives thereof used to inhibit the asexual reproduction of fungi can be varied by one skilled in the art over a wide range, depending on the agents utilization conditions.

The washing and/or cleaning agents according to the present invention contain preferably 0.000001 to 2, in particular 0.00001 to 1, particularly preferably 0.00001 to 0.1, especially 0.00005 to 0.05, in particular 0.00005 to 0.0005 wt % ortho-phenylphenol and/or derivatives thereof.

The agents according to the present invention are manufactured according to usual formulations known to one skilled in the art. Ortho-phenylphenol and/or derivatives thereof are preferably added to the agents once they are completely prepared, but can also be added during the manufacturing process if this is desirable.

Inhibition of the asexual reproduction of fungi on textiles or plastic surfaces often prevents a reinfection of body regions that were already previously affected. Inhibition of the asexual reproduction of fungi on ceramics, plastics, or metals reduces the risk of infection or reinfection without stressing the skin, mucous membranes, or wastewater with substances having fungicidal or fungicidal action. Catheters as well as other medical devices and/or prostheses manufactured from plastic or metals can likewise be kept largely free of fungi by the use of ortho-phenylphenol and/or derivatives thereof in, for example, rinses or cleaning agents.

According to a further particular embodiment, ortho-phenylphenol and/or derivatives thereof is added to washing and/or cleaning agents. Modern textile fibers in particular, which cannot be washed with full-strength washing agents or at high temperatures, cannot be completely cleansed of fungi by means of usual delicate fabric washing agents or at washing temperatures of 30 or 40° C. An advantage of the utilization of such substances usable according to the present invention in washing and cleaning agents is that despite a low wastewater impact and a low risk of creating resistance, garments can be kept free of sporulating fungi.

Ortho-phenylphenol and/or derivatives thereof can also be added according to the present invention to cleaning agents that are used for cleaning hard surfaces such as, for example, floors, tiles, floor tiles, plastics, and other hard surfaces in the home, in particular in high-humidity areas (e.g. bathrooms), or in clinical practice, where they can prevent the undesirable discoloration of surfaces due to the formation of colored spores (e.g. black, for Aspergillus niger). Shower curtains and other bath textiles, as well as plastics, can also be protected from mold-related discoloration.

“Washing and cleaning agents” are understood in the context of the present invention, in the widest sense, as surfactant-containing preparations in solid form (particles, powders, etc.), semi-solid form (pastes. etc.), liquid form (solutions, emulsions, suspensions, gels, etc.), and a form similar to gas (aerosols, etc.) that, in the interest of an advantageous effect upon application, contain a surfactant or multiple surfactants, usually in addition to further components that are usual for the respective purpose. Examples of such surfactant-containing preparations are surfactant-containing washing-agent preparations, surfactant-containing cleaning agents for hard surfaces, or surfactant-containing brightening-agent preparations that can in each case be solid or liquid, but can also be present in a form that encompasses solid or liquid components or partial quantities of the components alongside one another.

The washing and cleaning agents can contain ingredients that are usually contained, such as anionic, nonionic, cationic, and amphoteric surfactants, inorganic and organic builder substances, specific polymers (for example, those having co-builder properties), foam inhibitors, dyes, and optionally additional fragrances (perfumes), bleaching agents (such as, for example, peroxo bleaching agents and chlorine bleaching agents), bleach activators, bleach stabilizers, bleach catalysts, enzymes, and graying inhibitors, without limiting the ingredients to these groups of substances. Important ingredients of these preparations are often also washing adjuvants, which are to be understood by way of example, and without limitation, as optical brighteners, UV-protective substances, so-called soil repellents (i.e. polymers that counteract re-soiling of fibers). The individual substance groups are explained in further detail hereinafter.

For the case in which the preparations are present at least in part as shaped elements, binding and disintegration adjuvants can also be contained.

Anionic, nonionic, zwitterionic, and cationic surfactants can be used as surfactants.

Anionic surfactants that can be used are, for example, those of the sulfonate and sulfate types. Possibilities as surfactants of the sulfonate type are, preferably, C₉₋₁₃ alkyl benzenesulfonates, olefinsulfonates, i.e. mixtures of alkene- and hydroxyalkanesulfonates, and disulfonates, for example such as those obtained from C₁₂₋₁₈ monoolefins having an end-located or internal double bond, by sulfonation with gaseous sulfur trioxide and subsequent alkaline or acid hydrolysis of the sulfonation products. Also suitable are alkanesulfonates that are obtained from C₁₂₋₁₈ alkanes, for example by sulfochlorination or sulfoxidation with subsequent hydrolysis and neutralization. The esters of 2-sulfo fatty acids (estersulfonates), e.g. the 2-sulfonated methyl esters of hydrogenated coconut, palm kernel, or tallow, fatty acids, are likewise suitable.

Further suitable anionic surfactants are sulfonated fatty acid glycerol esters. “Fatty acid glycerol esters” are understood as the mono-, di- and triesters, and mixtures thereof, that are obtained during the production by esterification of a monoglycerol with 1 to 3 mol fatty acid, or upon transesterification of triglycerides with 0.3 to 2 mol glycerol. Preferred sulfonated fatty acid glycerol esters are the sulfonation products of saturated fatty acids having 6 to 22 carbon atoms, for example hexanoic acid, octanoic acid, decanoic acid, myristic acid, lauric acid, palmitic acid, stearic acid, or behenic acid.

Preferred alk(en)yl sulfates are the alkali, and in particular sodium, salts of the sulfuric acid semi-esters of the C₁₂-C₁₈ fatty alcohols, for example from coconut fatty alcohol, tallow alcohol, lauryl, myristyl, cetyl, or stearyl alcohol, or the C₁₀-C₂₀ oxo alcohols and those semi-esters of secondary alcohols of those chain lengths. Additionally preferred are alk(en)yl sulfates of the aforesaid chain length that contain a synthetic straight-chain alkyl radical produced on a petrochemical basis, which possess a breakdown behavior analogous to those appropriate compounds based on fat-chemistry raw materials. In washing and cleaning agents, the C₁₂-C₁₆ alkyl sulfates and C₁₂-C₁₅ alkyl sulfates, as well as C₁₄-C₁₅ alkyl sulfates, are preferred. 2,3-alkyl sulfates that are manufactured, for example, according to U.S. Pat. Nos. 3,234,258 or 5,075,041 and can be obtained, as commercial products of the Shell Oil Company, under the name DAN®, are also suitable anionic surfactants.

The sulfuric acid monoesters of straight-chain or branched C₇₋₂₁ alcohols ethoxylated with 1 to 6 mol ethylene oxide, such as 2-methyl-branched C₉₋₁₁ alcohols with an average of 3.5 mol ethylene oxide (EO) or C₁₂₋₁₈ fatty alcohols with 1 to 4 EO, are also suitable. Because of their high foaming characteristics they are used in washing and cleaning agents only in relatively small quantities, for example in quantities of 1 to 5 wt %.

Other suitable anionic surfactants are also the salts of alkylsulfosuccinic acid, which are also referred to as sulfosuccinates or as sulfosuccinic acid esters and represent the monoesters and/or diesters of sulfosuccinic acid with alcohols, preferably fatty alcohols, and in particular ethoxylated fatty alcohols. Preferred sulfosuccinates contain C₈₋₁₈ fatty alcohol radicals or mixtures thereof. Particularly preferred sulfosuccinates contain a fatty alcohol radical that is derived from ethoxylated fatty alcohols which, considered per se, represent nonionic surfactants (see below for description). Sulfosuccinates whose fatty alcohol radicals derive from ethoxylated fatty alcohols with a restricted homolog distribution are, in turn, particularly preferred. It is likewise possible to use alk(en)ylsuccinic acid having preferably 8 to 18 carbon atoms in the alk(en)yl chain, or salts thereof.

Further appropriate anionic surfactants are, in particular, soaps. Saturated fatty acid soaps, such as the salts of lauric acid, myristic acid, palmitic acid, stearic acid, hydrogenated erucic acid, and behenic acid, are suitable, as are soap mixtures derived in particular from natural fatty acids, e.g. coconut, palm kernel, or tallow fatty acids.

The anionic surfactants, including the soaps, can be present in the form of their sodium, potassium, or ammonium salts, and as soluble salts of organic bases, such as mono-, di-, or triethanolamine. The sodium or potassium salts, in particular the sodium salts, are preferred. The surfactants can likewise be used in the form of their magnesium salts.

Those agents that contain 5 to 50 wt %, preferably 7.5 to 40 wt %, and in particular 15 to 25 wt % of one or more anionic surfactant(s) are preferred in the context of the present invention.

The nonionic surfactants used are preferably alkoxylated, advantageously ethoxylated, in particular primary alcohols preferably having 8 to 18 carbon atoms and an average of 1 to 12 mol ethylene oxide (EO) per mol of alcohol, in which the alcohol radical can be linear or preferably methyl-branched in the 2-position, or can contain mixed linear or methyl-branched radicals, such as those usually present in oxo alcohol radicals. Particularly preferred, however, are alcohol ethoxylates having linear radicals made up of alcohols of natural origin having 12 to 18 carbon atoms, e.g. from coconut, palm, tallow, or oleyl alcohol, and an average of 2 to 8 EO per mol of alcohol. The preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO, or 8 EO, C₁₂₋₁₈ alcohols having 3 EO, 5 EO, or 7 EO, and mixtures thereof, such as mixtures of C₁₂₋₁₄ alcohol having 3 EO and C₁₂₋₁₆ alcohol having 5 EO. The degrees of ethoxylation that are indicated represent statistical averages, which for a specific product may be a whole or fractional number. Preferred alcohol ethoxylates exhibit a restricted homolog distribution (=narrow range ethoxylates, NRE). In addition to these nonionic surfactants, fatty alcohols having more than 12 EO can also be used. Examples of these are tallow alcohol having 14 EO, 25 EO, 30 EO, or 40 EO.

A further class of nonionic surfactants that are preferred for use, which can be used either as the only nonionic surfactant or in combination with other nonionic surfactants, are alkoxylated, preferably ethoxylated or ethoxylated and propoxylated, fatty acid alkyl esters preferably having 1 to 4 carbon atoms in the alkyl chain, in particular fatty acid methyl esters.

A further class of nonionic surfactants that can advantageously be used is the alkyl polyglycosides (APGs). Usable alkyl polyglycosides conform to the general formula RO(G)_{z}, in which R denotes a linear or branched, in particular methyl-branched in the 2-position, saturated or unsaturated aliphatic radical having 8 to 22, preferably 12 to 18 C atoms, and G is the symbol that denotes a glycose unit having 5 or 6 C atoms, preferably glucose. The glycosidation number z is between 1.0 and 4.0, preferably between 1.0 and 2.0 and in particular between 1.1 and 1.4.

Linear alkyl polyglucosides, i.e. alkyl polyglycosides in which the polyglycosyl radical is a glucose radical and the alkyl radical is an n-alkyl chain, are preferably used.

The surfactant-containing preparations according to the present invention can preferably contain alkyl polyglycosides, APG contents in the preparations provided for washing, dishwashing, or cleaning purposes of over 0.2 wt %, based on the entire preparation, being preferred. Particularly preferred surfactant-containing preparations contain APG in quantities from 0.2 to 10 wt %, preferably in quantities from 0.2 to 5 wt %, and in particular in quantities from 0.5 to 3 wt %.

Nonionic surfactants of the amine oxide type, for example N-cocalkyl-N,N-dimethylamine oxide and N-tallowalkyl-N,N-dihydroxyethylamine oxide, and the fatty acid alkanolamides, can also be suitable. The quantity of these nonionic surfactants is preferably no more than that of the ethoxylated fatty alcohols, in particular no more than half thereof.

Further suitable surfactants are polyhydroxy fatty acid amides of formula (V) 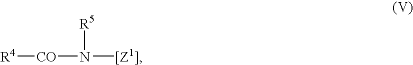
in which R⁴CO denotes an aliphatic acyl radical having 6 to 22 carbon atoms; R⁵ denotes hydrogen, an alkyl or hydroxyalkyl radical having 1 to 4 carbon atoms, and [Z¹] denotes a linear or branched polyhydroxyalkyl radical having 3 to 10 carbon atoms and 3 to 10 hydroxyl groups. The polyhydroxy fatty acid amides are known substances that can usually be obtained by reductive amination of a reducing sugar with ammonia, an alkylamine, or an alkanolamine, and subsequent acylation with a fatty acid, a fatty acid alkyl ester, or a fatty acid chloride.

Also belonging to the group of the polyhydroxy fatty acid amides are compounds of formula (VI) 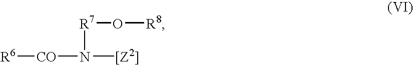
in which R⁶ denotes a linear or branched alkyl or alkenyl radical having 7 to 12 carbon atoms; R⁷ denotes a linear, branched, or cyclic alkyl radical or an aryl radical having 2 to 8 carbon atoms; and R⁸ denotes a linear, branched, or cyclic alkyl radical or an aryl radical or an oxyalkyl radical having 1 to 8 carbon atoms C₁₋₄ alkyl or phenyl radicals being preferred; and [Z²] denotes a linear polyhydroxyalkyl radical whose alkyl chain is substituted with at least two hydroxyl groups, or alkoxylated, preferably ethoxylated or propoxylated, derivatives of that radical.

[Z²] is preferably obtained by reductive amination of a reduced sugar, for example glucose, fructose, maltose, lactose, galactose, mannose, or xylose. The N-alkoxy- or N-aryloxy-substituted compounds can then be converted, as described in WO-A-95/07331, into the desired polyhydroxy fatty acid amides by reaction with fatty acid methyl esters in the presence of an alkoxide as catalyst.

It can also be preferred to use cationic surfactants in addition to anionic and nonionic surfactants.

Cationic surfactants may be mentioned in particular as textile-softening substances. Examples of cationic surfactants are, in particular, quaternary ammonium compounds, cationic polymers, and emulsifiers.

Suitable examples are quaternary ammonium compounds of formulas (VII) and (VIII) 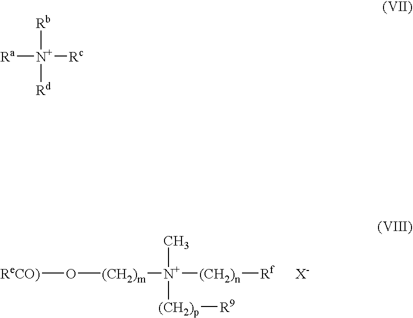
where in (VII), R^{a} and R^{b} denote an acyclic alkyl radical having 12 to 24 carbon atoms; R^{c} denotes a saturated C₁-C₄ alkyl or hydroxyalkyl radical; and R^{d} either is identical to R^{a}R^{b}, or R^{c} or denotes an aromatic radical. X′″ denotes either a halide, methosulfate, methophosphate, or phosphate ion, and mixtures thereof. Examples of such cationic compounds are didecyldimethylammonium chloride, ditallowdimethylammonium chloride, or dihexadecylammonium chloride.

Compounds of formula (VIII) are so-called esterquats. Esterquats are characterized by outstanding biodegradability. Here R^{e} denotes an aliphatic alkyl radical having 12 to 22 carbon atoms with 0, 1, 2, or 3 double bonds; R^{f} denotes H, OH, or O(CO)R^{h}; and R^{g} denotes, independently of R^{f}, H, OH, or O(CO)Rⁱ, R^{h} and Rⁱ each denoting, independently of one another, an aliphatic acyl radical having 12 to 22 carbon atoms with 0, 1, 2, or 3 double bonds m, n, and p can each, independently of one another, have a value of 1, 2, or 3. X′″ can be either a halide, methosulfate, methophosphate, or phosphate ion, as well as mixtures thereof. Compounds that contain the group O(CO)R^{h} for R^{f}, and alkyl radicals having 16 to 18 carbon atoms for R^{c} and R^{h}, are preferred. Compounds in which R^{g} additionally denotes OH are particularly preferred. Examples of compounds of formula (VIII) are methyl-N-(2-hydroxyethyl)-N,N-di(tallowacyloxyethyl)ammonium methosulfate, bis-(palmitoyl)ethylhydroxyethylmethylammonium methosulfate, or methyl-N,N-bis(acyloxyethyl)-N-(2-hydroxyethyl)ammonium methosulfate. If quaternized compounds of formula (VIII) having unsaturated alkyl chains are used, those acyl groups whose corresponding fatty acids have an iodine number between 5 and 80, preferably between 10 and 60, and in particular between 15 and 45, and that have a cis/trans isomer ratio (in wt %) greater than 30:70, preferably greater than 50:50, and in particular greater than 70:30, are preferred. Commercial examples are the methylhydroxyalkyldialkoyl oxyalkylammonium methosulfates marketed by Stepan under the trade name Stepantex®, or the products of Cognis known as Dehyquat®, or the products of Goldschmidt-Witco known as Rewoquat®. Further preferred compounds are the diesterquats of formula (IX) that are obtainable under the name Rewoquat® W 222 LM or CR 3099, and provide not only softness but also stability and color protection.

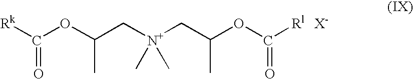

R^{k} and R^{l} here each denote, independently of one another, an aliphatic radical having 12 to 22 carbon atoms with 0, 1, 2, or 3 double bonds.

In addition to the quaternary compounds described above, other known compounds for example quaternary imidazolinium compounds of formula (X) can also be used. 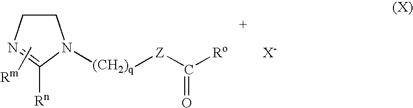
in which R^{m} denotes H or a saturated alkyl radical having 1 to 4 carbon atoms; Rⁿ and R^{o} each, independently of one another, denote an aliphatic, saturated, or unsaturated alkyl radical having 12 to 18 carbon atoms; Rⁿ can alternatively also denote O(CO)R^{p}, where R^{p} signifies an aliphatic, saturated, or unsaturated alkyl radical having 12 to 18 carbon atoms; Z signifies an NH group or oxygen; and X′″ is an anion. q can assume integer values between 1 and 4.

Further suitable quaternary compounds are described by formula (XI), 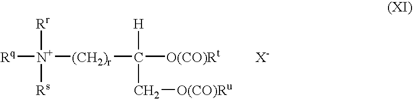
in which R^{q}, R^{r}, and R^{s}, independently of one another, denote a C₁₋₄ alkyl, alkenyl, or hydroxyalkyl group, R^{t} and R^{u}, each selected independently, represent a C₈₋₂₈ alkyl group; and r is a number between 0 and 5.

In addition to the compounds of formulas (V) to (IX), short-chain water-soluble quaternary ammonium compounds can also be used, such as trihydroxyethylmethylammonium methosulfate or the alkyltrimethylammonium chlorides, dialkyldimethylammonium chlorides, and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride.

Also suitable are protonated alkylamine compounds that have a softening effect, as well as unquaternized protonated precursors of the cationic emulsifiers.

The quaternized protein hydrolysates represent further cationic compounds usable according to the present invention.

Suitable cationic polymers include the Polyquaternium polymers such as those described in the CTFA Cosmetic Ingredient Dictionary (The Cosmetic Toiletry and Fragrance Association, Inc., 1997), in particular the Polyquaternium-6, Polyquaternium-7, and Polyquaternium-10 polymers also referred to as merquats (Ucare Polymer IR 400; Amerchol), Polyquaternium-4 copolymers such as graft copolymers having a cellulose skeleton and quaternary ammonium groups that are bound via allyldimethylammonium chloride, cationic cellulose derivatives such as cationic guar, such as guar hydroxypropyltriammonium chloride, and similar quaternized guar derivatives (e.g. Cosmedia Guar, manufacturer, Cognis GmbH), cationic quaternary sugar derivatives (cationic alkyl polyglucosides). e.g. the commercial product Glucquat® 100% according to CTFA nomenclature a “lauryl methyl gluceth-10 hydroxypropyl dimonium chloride,” copolymers of PVP and dimethyl aminomethacrylate, copolymers of vinylimidazole and vinylpyrrolidone, aminosilicone polymers and copolymers.

Also usable are polyquaternized polymers (e.g. Luviquat Care of BASE) and also chitin-based cationic biopolymers and derivatives thereof, for example the polymer obtainable under the commercial name Chitosan® (manufacturer: Cognis).

Likewise suitable according to the present invention are cationic silicone oils, for example the commercially available products Q2-7224 (manufacturer: Dow Corning; a stabilized trimethylsilylamodimethicone), Dow Corning 929 Emulsion (containing a hydroxylamino-modified silicone that is also referred to as amodimethicone), SM-2059 (manufacturer, General Electric), SLM-55067 (manufacturer: Wacker), Abil®-Quat 3270 and 3272 (manufacturer: Goldschmidt-Rewo; diquaternary polydimethylsiloxanes. quaternium-80), and the siliconequat Rewoquat® SQ 1 (Tegopren® 6922, manufacturer: Goldschmidt-Rewo).

Also usable are compounds of formula (XII), 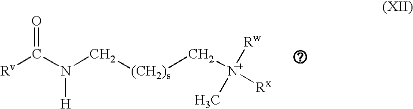
which can be alkylamidoamines in their unquaternized or, as depicted, quaternized form. R^{v} can be an aliphatic acyl radical having 12 to 22 carbon atoms with 0, 1, 2, or 3 double bonds. s can assume values between 0 and 5. R^{w} and R^{x} each denote, independently of one another, H, C₁₋₄ alkyl, or hydroxyalkyl. Preferred compounds are fatty acid aminoamines such as the stearylaminopropyldimethylamine obtainable under the name Tego Amid® S 18, or the 3-tallowamidopropyltrimethylammonium methosulfate obtainable under the name Stepantex® X 9124, which are distinguished not only by a good conditioning action but also by a color transfer-inhibiting effect, and especially by their good biodegradability.

If cationic surfactants are used, they are contained in the preparations preferably in quantities from 0.01 to 10 wt %, in particular 0.1 to 3.0 wt %.

The total surfactant content in the agents according to the present invention can be between 5 and 50 wt %, preferably between 10 and 35 wt %.

In addition to the surfactants, detergency builders are the most important ingredients of washing and cleaning agents. Detergency builders usually used in washing and cleaning agents, i.e. in particular zeolites, silicates carbonates, organic co-builders and even (where no environmental prejudices against their use exist) phosphates, can be contained in the surfactant-containing preparations according to the present invention.

Suitable crystalline, sheet-form sodium silicates possess the general formula NaMSiₓO₂ₓ₊₁·H₂O, where M denotes sodium or hydrogen, x a number from 1.9 to 4, and y is a number from 0 to 20, and preferred values for x are 2, 3, or 4. Crystalline sheet silicates of this kind are described, for example, in European Patent Application EP-A-0 164 514. Preferred crystalline sheet silicates of the formula indicated above are those in which M denotes sodium and x assumes the value 2 or 3. Both β- and δ-sodium disilicates Na₂Si₂O₅·yH₂O are particularly preferred, β-sodium disilicate can be obtained, for example, according to the method described in International Patent Application WO-A-91/08171.

Also usable are amorphous sodium silicates having a Na₂O:SiO₂ modulus of 1:2 to 1:3.3, preferably 1:2 to 1:2.8, and in particular 1:2 to 1:2.6, which are dissolution-delayed and exhibit secondary washing properties. Dissolution delay as compared with conventional amorphous sodium silicates can have been brought about in various ways, for example by surface treatment, compounding, compacting/densification, or overdrying. So-called X-amorphous silicates, which also exhibit a dissolution delay as compared with conventional water glasses, are described, for example, in German Patent Application DE-A-44 00 024. The products exhibit microcrystalline regions 10 to a few hundred nm in size, values up to a maximum of 50 nm and in particular up to a maximum of 20 nm being preferred. Densified/compacted amorphous silicates, compounded amorphous silicates, and overdried X-amorphous silicates are particularly preferred.

A finely crystalline synthetic zeolite containing bound water that is used if applicable is preferably zeolite A and/or zeolite P. Zeolite MAP® (e.g. the commercial product Doucil A 24 of the Crosfield Co.) is particularly preferred as a P-type zeolite. Also suitable, however, are zeolite X as well as mixtures of A, X, and/or P. Also commercially available and preferred for use in the context of the present invention is, for example, a co-crystal of zeolite X and zeolite A (approx. 80 wt % zeolite X) that is marketed by CONDEA Augusta S.p.A. under the trade name VEGOBOND AX® and can be described by the formula
nNa₂·(1−n)K₂O·Al₂O₃·(2−2.5)SiO₂·(3.5−5.5)H₂O.
Suitable zeolites exhibit an average particle size of less than 10 μm (volume distribution; measurement method: Coulter Counter), and preferably contain 18 to 22 wt %, in particular 20 to 22 wt %, bound water.

Use of the commonly known phosphates as builder substances is also possible, of course, provided such use is not to be avoided for environmental reasons. The sodium salts of the orthophosphates, pyrophosphates, and in particular tripolyphosphates are particularly suitable.

Usable organic builder substances are, for example, the polycarboxylic acids usable in the form of their sodium salts, “polycarboxylic acids” being understood as those carboxylic acids that carry more than one acid function. These are, for example, citric acid, adipic acid, succinic acid, glutaric acid, malic acid, tartaric acid, maleic acid, fumaric acid, sugar acids, aminocarboxylic acids, nitrilotriacetic acid (NTA), provided such use is not objectionable for environmental reasons, as well as mixtures thereof. Preferred salts are the salts of the polycarboxylic acids such as citric acid, adipic acid, succinic acid, glutaric acid, tartaric acid, sugar acids, and mixtures thereof. The acids per se can also be used. The acids typically also possess, in addition to their builder effect, the property of an acidifying component, and thus serve also to establish a lower and milder pH for detergents or cleaning agents. To be mentioned in this context are citric acid, succinic acid, glutaric acid, adipic acid, gluconic acid, and any mixtures thereof.

Polymeric polycarboxylates are also suitable as builders. These are for example, the alkali-metal salts of polyacrylic acid or polymethacrylic acid, for example those having a relative molecular weight of 500 to 70,000 g/mol.

The molar weights indicated for the polymeric polycarboxylates are, in the context of the present invention, weight-averaged molar weights M_{w} of the respective acid form that were determined in principle by means of gel permeation chromatography (GPC), a UV detector having been used. The measurement was performed against an external polyacrylic acid standard that, because of its structural affinity with the polymers being investigated, yields realistic molecular weight values. These indications deviate considerably from the molecular weight indications in which polystyrenesulfonic acids are used as the standard. The molar weights measured against polystyrenesulfonic acids are usually much higher than the molar weights indicated in the context of the present invention.

Suitable polymers are, in particular, polyacrylates that preferably have a molecular weight from 2000 to 20,000 g/mol. Because of their superior solubility, of this group the short-chain polyacrylates that have molar weights from 2000 to 10,000 g/ml, and particularly preferably from 3000 to 5000 g/mol, may in turn be preferred.

Copolymeric polycarboxylates, in particular those of acrylic acid with methacrylic acid or of acrylic acid or methacrylic acid with maleic acid, are also suitable. Copolymers of acrylic acid with maleic acid that contain 50 to 90 wt % acrylic acid and 50 to 10 wt % maleic acid have proven particularly suitable. Their relative molecular weight, based on free acids, is generally 2000 to 70,000 g/mol, preferably 20,000 to 50,000 g/mol, and in particular 30,000 to 40,000 g/mol.

The (co)polymeric polycarboxylates can be used as either a powder or an aqueous solution. The (co)polymeric polycarboxylate content of the washing and cleaning agents according to the present invention is preferably 0.5 to 20 wt %, in particular 3 to 10 wt %.

To improve water solubility, the polymers can also contain allylsulfonic acids, allyloxybenzenesulfonic acid, and methallylsulfonic acid as monomers.

Also particularly preferred are biodegradable polymers made up of more than two different monomer units for example those that contain salts of acrylic acid and of maleic acid, as well as vinyl alcohol or vinyl alcohol derivatives, as monomers, or that contain salts of acrylic acid and of 2-alkylallylsulfonic acid, as well as sugar derivatives, as monomers.

Additionally preferred copolymers are those that contain preferably acrolein and acrylic acid/acrylic acid salts or acrolein and vinyl acetate, as monomers.

Also to be mentioned as further preferred builder substances are polymeric aminodicarboxylic acids, their salts, or their precursor substances. Particularly preferred are polyaspartic acids and their salts and derivatives, some of which have not only co-,builder properties but also a bleach-stabilizing effect.

Other suitable builder substances are polyacetals, which can be obtained by reacting dialdehydes with polyolcarboxylic acids that have 5 to 7 carbon atoms and at least 3 hydroxy groups. Preferred polyacetals are obtained from dialdehydes such as glyoxal, glutaraldehyde, terephthaladehyde, and mixtures thereof, and from polyolcarboxylic acids such as gluconic acid and/or glucoheptonic acid.

Other suitable organic builder substances are dextrins, for example oligomers or polymers of carbohydrates, which can be obtained by partial hydrolysis of starches. The hydrolysis can be performed in accordance with usual, e.g. acid- or enzyme-catalyzed, methods. Preferably these are hydrolysis products having average molar weights in the range from 400 to 500,000 g/mol. A polysaccharide having a dextrose equivalent (DE) in the range from 0.5 to 40, in particular from 2 to 30, is preferred, DE being a common indicator of the reducing effect of a polysaccharide as compared with dextrose, which possesses a DE of 100. Both maltodextrins having a DE between 3 and 20 and dry glucose syrups having a DE between 20 and 37, and so-called yellow dextrins and white dextrins having higher molar weights in the range from 2000 to 30,000 g/mol, are usable. A preferred dextrin is described in GB Patent Application 94 19 091.

The oxidized derivatives of such dextrins are their reaction products with oxidizing agents that are capable of oxidizing at least one alcohol function of the saccharide ring to the carboxylic acid function. An oxidized oligosaccharide is likewise suitable; a product oxidized at C₆ of the saccharide ring can be particularly advantageous.

Oxydisuccinates and other derivatives of disuccinates, preferably ethylenediamine disuccinate, are also additional suitable co-builders. Ethylenediamine N,N′-disuccinate (EDDS) is used here preferably in the form of its sodium or magnesium salts. Also preferred in this context are glycerol disuccinates and glycerol trisuccinates. Suitable utilization quantities in zeolite-containing and/or silicate-containing formulations are 3 to 15 wt %.

Other usable organic co-builders are, for example, acetylated hydroxycarboxylic acids and their salts, which can optionally also be present in lactone form and which contain at least 4 carbon atoms and at least one hydroxy group, as well as a maximum of two acid groups.

A further substance class having co-builder properties is represented by the phosphonates. These are, in particular, hydroxyalkane- and aminoalkanephosphonates. Among the hydroxyalkanephosphonates, 1-hydroxyethane-1,1-diphosphonate (HEDP) is particularly important as a co-builder. It is preferably used as a sodium salt, in which context the disodium salt reacts neutrally and the tetrasodium salt in alkaline fashion (pH 9). Suitable aminoalkanephosphonates are preferably ethylenediamine tetramethylenephosphonate (EDTMP), diethylenetriamine pentamethylenephosphonate (DTPMP), and their higher homologs. They are preferably used in the form of the neutrally reacting sodium salts, e.g. as a hexasodium salt of EDTMP or as a hepta- and octasodium salt of DTPMP. Of the class of phosphonates, HEDP is preferably used as a builder. The aminoalkanephosphonates furthermore possess a pronounced heavy-metal binding capability. It may accordingly be preferred, especially when the surfactant-containing preparations according to the present invention also contain bleaches, to use aminoalkanephosphonates, in particular DTPMP, or mixtures of the aforesaid phosphonates.

All compounds that are capable of forming complexes with alkaline-earth metal ions can furthermore be used as co-builders.

Of the compounds yielding H₂O₂ in water that serve as bleaching agents, sodium perborate tetrahydrate, and sodium perborate monohydrate are of particular importance. Other usable bleaching agents are, for example, sodium percarbonate, peroxypyrophosphates, citrate perhydrates, and peracid salts or peracids that yield H₂O₂, such as perbenzoates, peroxophthalates, diperazelaic acid, phthaloimino peracid, or diperdodecanedioic acid. If cleaning or bleaching-agent preparations for automatic dishwashing are being produced, bleaching agents from the group of the organic bleaching agents can also be used. Typical organic bleaching agents are the diacyl peroxides, for example dibenzoyl peroxide. Further typical organic bleaching agents are the peroxy acids, the alkylperoxy acids and arylperoxy acids being mentioned in particular as examples. Preferred representatives are (a) peroxybenzoic acid and its ring-substituted derivatives, such as alkylperoxybenzoic acids but also peroxy-α-naphthoic acid and magnesium monoperphthalate, (b) the aliphatic or substituted aliphatic peroxy acids, such as peroxylauric acid, peroxystearic acid, ε-phthalimidoperoxycaproic acid [phthaloiminoperoxyhexanoic acid (PAP)], o-carboxybenzamidoperoxycaproic acid, N-nonenylamidoperadipic acid, and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, the diperoxyphthalic acids, 2-decyldiperoxybutane-1,4-dioic acid, and N,N-terephthaloyl-di(6-aminopercaproic) acid.

In order to achieve an improved bleaching effect when washing or cleaning at temperatures of 60° C. and below, bleach activators can be incorporated into the surfactant-containing preparations. Compounds that under perhydrolysis conditions, yield aliphatic peroxocarboxylic acids having preferably 1 to 10 carbon atoms, in particular 2 to 4 carbon atoms, and/or optionally substituted perbenzoic acids, can be used as bleach activators. Substances that carry O- and/or N-acyl groups having the aforesaid number of carbon atoms, and/or optionally substituted benzoyl groups, are suitable. Multiply acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl or isononanoyloxybenzenesulfonate (n- or iso-NOBS), carboxylic acid anhydrides, in particular phthalic acid anhydride, acylated polyvalent alcohols, in particular triacetin, ethylene glycol diacetate, and 2,5-diacetoxy-2,5-dihydrofuran, are preferred.

In addition to or instead of the conventional bleach activators, so-called bleach catalysts can also be incorporated into the surfactant-containing preparations. These substances are bleach-intensifying transition-metal salts or transition-metal complexes such as, for example, Mn, Fe, Co, Ru, or Mo salt complexes or carbonyl complexes. Mn, Fe, Co, Ru, Mo, Ti, V, and Cu complexes having nitrogen-containing tripod ligands, as well as Co, Fe, Cu, and Ru ammine complexes, are also usable as bleach catalysts.

Suitable enzymes are, in particular, those of the protease, esterases, lipase, amylase, cellulase classes, and mixtures thereof. Enzymatic active substances obtained from bacterial strains or fungi, such as Bacillus subtilis, Bacillus licheniformis, and Streptomyceus griseus, are particularly suitable. Proteases of the subtilisin type, and in particular proteases obtained from Bacillus lentus, are preferably used. Enzyme mixtures, for example of protease and amylase or protease and lipase or protease and cellulase, or of cellulase and lipase, or of protease, amylase, and lipase or protease, lipase, and cellulase, but in particular cellulase-containing mixtures, are of particular interest in this context. Peroxidases or oxidases have also proven suitable in certain cases. The enzymes can be adsorbed onto carrier materials and/or embedded in encasing substances, in order to protect them from premature breakdown. The proportion of enzymes, enzyme mixtures, or enzyme granules in the surfactant-containing preparations according to the present invention can be, for example, approximately 0.1 to 5 wt %, preferably 0.1 to approximately 2 wt %.

Optical brighteners are a preferred group of suitable additives. The optical brighteners usual in washing agents can be used here. Examples of optical brighteners are derivatives of diaminostilbenesulfonic acid or its alkali-metal salts. Suitable, for example, are salts of 4,4′-bis(2-anilino-4-morpholino-1,3,5-triazinyl-6-amino)stilbene-2,2′-disulfonic acid, or compounds of similar structure that carry, instead of the morpholino group, a diethanolamino group, a methylamino group, an anilino group, or a 2-methoxyethylamino group. Brighteners of the substituted diphenylstyryl type can also be present in the sub-portions (preparations having washing activity) of the surfactant-containing preparations according to the present invention, e.g. the alkali salts of 4,4′-bis(2-sulfostyryl)diphenyl, of 4,4′-bis(4-chloro-3-sulfostyryl)diphenyl, or of 4-(4-chlorostyryl)-4′-(2-sulfostyryl)diphenyl. Mixtures of the aforesaid brighteners can also be used.

A further group of additives that is preferred according to the present invention is UV-protection substances. UV absorbers can be absorbed onto the treated textiles and improve the light resistance of the fibers and/or the light resistance of the other formulation constituents. “UV absorbers” are understood as organic substances (light protection filters) that are capable of absorbing ultraviolet radiation and re-emitting the absorbed energy in the form of longer-wave radiation, e.g. heat. Compounds that exhibit these desired properties are, for example, the compounds and derivatives of benzophenone, with substituents in the 2- and/or 4-position, that are effective by radiationless deactivation. Also suitable are substituted benzotriazoles, for example water-soluble benzenesulfonic acid-3-(2H-benzotriazol-2-yl)-4-hydroxy-5-(methylpropyl)monosodium salt (Cibafast® H), acrylates phenyl-substituted in the 3-position (cinnamic acid derivatives), optionally with cyano groups in the 2-position, salicylates, organic Ni complexes, and natural substances such as umbelliferon and body-derived urocanic acid. Particularly important are biphenyl derivatives and especially stilbene derivates, such as those described e.g. in EP 0728749 A and available commercially as Tinosorb® FD or Tinosorb® FR from Ciba. To be mentioned as UV-B absorbers are 3-benzylidene camphor and 3-benzylidene norcamphor and its derivatives, e.g. 3-(4-methylbenzylidene)camphor, as described in EP 0693471 B1; 4-aminobenzoic acid derivatives, preferably 4-(dimethylamino)benzoic acid 2-ethylhexyl ester, 4-(dimethylamino)benzoic acid 2-octyl ester, and 4-(dimethylamino)benzoic acid amyl ester; esters of cinnamic acid, preferably 4-methoxycinnamic acid 2-ethylhexyl ester, 4-methoxycinnamic acid propyl ester, 4-methoxycinnamic acid isoamyl ester, 2-cyano-3,3-phenylcinnamic acid 2-ethylhexyl ester(octocrylene); esters of salicylic acid, preferably salicylic acid 2-ethylhexyl ester, salicylic acid 4-isopropylbenzyl ester, salicylic acid homomenthyl ester; benzophenone derivatives, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably 4-methoxybenzalmalonic acid di-2-ethylhexyl ester; triazine derivatives, for example 2,4,6-trianilino-(p-carbo-2′-ethyl-1′-hexyloxy)-1,3,5-triazine and octyl triazone as described in EP 0818450 A1, or dioctyl butamido triazone (Uvasorb® HEB); propane-1,3-diones, for example 1-(4-tert-butylphenyl)-3-(4′-methoxyphenyl)propane-1,3-dione; ketotricyclo(5.2.1.0)decane derivatives, such as those described in EP 0694521 B1. Also suitable are 2-phenylbenzimidazole-5-sulfonic acid and its alkali, alkaline-earth, ammonium, alkylammonium, alkanolammonium, and glucammonium salts; sulfonic acid derivatives of benzophenones, preferably 2-hydroxy-4-methoxybenzophenone-5-sulfonic acid and its salts; sulfonic acid derivatives of 3-benzylidene camphor, for example 4-(2-oxo-3-bornylidenemethyl)benzenesulfonic acid and 2-methyl-5-(2-oxo-3-bornylidene)sulfonic acid and its salts.

Typical UV-A filters that are suitable are, in particular, derivatives of benzoyl methane, for example 1-(4′-tert-butylphenyl)-3-(4′-methoxyphenyl propane-1,3-dione, 4-tert-butyl-4′-methoxydibenzoyl methane (Parsol 1789), 1-phenyl-3-(4′-isopropylphenyl)propane-1,3-dione, and enamine compounds as described in DE 19712033 A1 (BASF). The UV-A and UV-B filters can, of course, also be used in mixtures. In addition to the aforementioned soluble substances, insoluble light-protection pigments, namely finely dispersed, preferably nanoized metal oxides or salts, are also possible for this purpose. Examples of suitable metal oxides are, in particular, zinc oxide and titanium oxide, and also oxides of iron, zirconium, silicon, manganese, aluminum, and cerium, as well as mixtures thereof. Silicates (talc), barium sulfate, or zinc stearate can be used as salts. The oxides and salts are already used in the form of pigments for skin-care and skin-protection emulsions and decorative cosmetics. The particles should have an average diameter of less than 100 nm, preferably between 5 and 50 nm, and in particular between 15 and 30 nm. They can have a spherical shape, but particles of this kind that possess an ellipsoidal shape, or one otherwise deviating from the spherical conformation, can also be used. The pigments can also be present in surface-treated form, i.e. hydrophilized or hydrophobized. Typical examples are coated titanium dioxides, for example titanium dioxide T 805 (Degussa) or Eusolex® T2000 (Merck). Suitable as hydrophobic coating agents are chiefly silicones and especially trialkoxyoctylsilanes or simethicones. Micronized zinc oxide is preferably used. Further suitable UV light protection filters may be inferred from the overview by P. Finkel in SOFW-Journal 122, 543 (1996).

The UV absorbers are usually used in quantities from 0.01 wt % to 5 wt %, preferably 0.03 wt % to 1 wt %.

A further group of additives that is preferred according to the present invention is dyes, in particular water-soluble or water-dispersible dyes, Dyes that are ordinarily used to improved the visual product appeal in washing, dishwashing, cleaning, and brightening agents are preferred here. The selection of such dyes will present no difficulty to one skilled in the art, in particular since such usual dyes possess excellent shelf stability and insensitivity to the other ingredients of the preparations having washing activity and to light, and no pronounced substantivity with respect to textiles, in order not to color them. According to the present invention, the dyes are added to the washing and/or cleaning agents according to the invention in quantities of less than 0.01 wt %.

A further class of additives that can be added, according to the present invention, to the washing and/or cleaning agents is polymers. Suitable among these polymers are, on the one hand, polymers that exhibit co-builder properties in the context of washing and cleaning or dishwashing, i.e. for example polyacrylic acids, as well as modified polyacrylic acids or corresponding copolymers. A further group of polymers is polyvinylpyrrolidone and other graying inhibitors, such as copolymers of polyvinylpyrrolidone, cellulose ethers, and the like. Also preferred as possible polymers are so-called soil repellents, as described in detail below.

The washing and cleaning agents can also contain, as further additives according to the present invention, so-called soil repellents, i.e. polymers that are absorbed onto fibers, positively influence the ability of oils and fats to be washed out of textiles, and thus specifically counteract re-soiling. This effect becomes particularly apparent when the soiled textile is one that has already been previously washed several times with a washing or cleaning agent according to the present invention that contains this oil- and fat-dissolving component. The preferred oil- and fat-dissolving components include, for example, nonionic cellulose ethers such as methyl cellulose and methylhydroxypropyl cellulose having a 15 to 30 wt% proportion of methoxy groups and a 1 to 15 wt % proportion of hydroxypropoxy groups, based in each case on the nonionic cellulose ethers, as well as polymers, known from the existing art, of phthalic acid and/or terephthalic acid and of their derivatives, in particular polymers of ethylene terephthalates and/or polyethylene glycol terephthalates or anionically and/or nonionically modified derivatives thereof. Of these, the sulfonated derivates of phthalic acid polymers and terephthalic acid polymers are particularly preferred.

Especially when the preparations are liquid or gelled, they can also contain solvents. Examples of suitable solvents are univalent or polyvalent alcohols having 1 to 4 C atoms. Preferred alcohols are ethanol, 1,2-propanediol, glycerol, and any mixtures thereof. The solvents can be contained in liquid preparations in a quantity from 2 to 12 wt %, in particular between approximately 1 and 5 wt %, based on the completed preparation.

The aforesaid additives are added to the washing and/or cleaning agents in quantities up to at most 30 wt %, preferably 2 to 20 wt %.

According to a particular embodiment, liquid or solid washing agents are especially preferred. Likewise especially preferred are washing and cleaning agents that are suitable for delicate fabrics or gentle treatment of sensitive textiles.

This listing of washing- and cleaning-agent ingredients that can occur in the washing, dishwashing, or cleaning agents according to the present invention is by no means exhaustive, but merely reproduces the essential typical ingredients of such agents. In particular in cases in which the preparations are liquid or gelled, organic solvents can also be contained in the agents. These are preferably univalent or polyvalent alcohols having 1 to 4 C atoms. Preferred alcohols in such agents are ethanol, 1,2-propanediol, glycerol, and mixtures of these alcohols, in preferred embodiments, agents of these kinds contain 2 to 12 wt % of such alcohols. A result that is particularly favorable in general in the context of cleaning agents for hard surfaces is obtained when the weight ratio of surfactant to alcohol in the solution is between approximately 1:1.5 and approximately 2:1.

Cleaning agents for hard surfaces that can be applied in foaming or non-foaming form onto the surfaces are likewise particularly preferred. Advantageously, it is thereby possible to decrease or prevent, in high-humidity areas, the propagation of mold spores in indoor air and the spread of discolorations that are attributable to mold spores,

In addition to the aforementioned constituents, the aqueous liquids utilized according to the present invention can contain small quantities of further active substances and additives that are usual in cleaners for hard surfaces. Examples of such active substances are lime-dissolving organic acids such as citric acid, acetic acid, or lactic acid or water-soluble salts thereof, which are preferably contained in quantities of 2 to 6 wt %, based on the entire aqueous liquid, in said liquid.

It can be preferred to use a cleaning agent that is applied as a foam onto the surface to be cleaned, and remains there longer as a result. The cleaning effect can thus be greatly enhanced. Foam generation preferably occurs directly upon emergence of the liquid from the spray devices. In the case of manual spray pumps this is achieved by a particular configuration of the spray head which ensures that the aqueous liquid emerging from the spray nozzle is mixed with so much air that the liquid already strikes the surface as a foam. Correspondingly configured spray pumps are commercially usual. In the case of utilization as an aerosol, provision must be made, by suitable configuration of the spray mechanism in consideration of the composition of the cleaning liquid, that sufficient quantities of propellant gas always emerge together with the liquid, and then result in foaming of the liquid. If applicable, shaking must occur before utilization. The corresponding configuration of the aerosol container, suction tube, and valve is part of the routine activity of one skilled in the art, and will therefore not be described here in further detail. The quantity of liquid sprayed onto the surface to be cleaned in the course of the cleaning procedure is usually between approximately 10 g and approximately 60 g/m², in particular 20 g to 40 g/m². The foam is, usefully, distributed as evenly as possible over the surface to be cleaned, and can then automatically exert its cleaning action. Preferably, however, the surfaces are then wiped off with a moistened cloth or a sponge, the cloth or sponge being rinsed out from time to time with clean water in the case of larger surfaces. The treated surfaces can, of course, also be rinsed off with water, but this is generally not necessary, since the remaining cleaning-agent residues are entirely transparent when dry and remain practically invisible.

The Examples that follow are intended to explain the invention without, however, limiting it thereto.

### Exemplifying Embodiments

### EXAMPLE 1

### Effect of Ortho-Phenylphenol (OPP) on the Sporulation of Aspergillus niger

The surfaces of wort agar plates were each contaminated with 100 μl of a germ suspension (10³ CFU/ml) of Aspergillus niger (DSM1988). Different quantities of active substance (solutions in ethanol, see table for final concentrations) had previously been added to the agar plates. The plates were incubated for 3 days at 25° C. Sporulation was assessed visually, and the sporulation rate (in %) was determined. Unless otherwise indicated in the tables below, the concentrations of active substance that were used did not prevent growth of the test strain. A growth-inhibiting or germ-killing effect occurred only at a concentration of 0.05 wt % OPP. Sporulation, however, was already completely suppressed at an OPP concentration fifty times lower, with no occurrence of a growth-inhibiting effect.

TABLE 1

| | | | | | | | | | | |
|---|---|---|---|---|---|---|---|---|---|---|
| OPP concentration [%] | 0 | 0.00001 | 0.00005 | 0.0001 | 0.0005 | 0.001 | 0.005 | 0.05 | 0.1 | 1.0 |
| Sporulation [%] | 100 | 95 | 95 | 85 | 10 | 0 | 0 | no growth | no growth | no growth |

Sporulation was inhibited with increasing concentrations, and completely suppressed at a utilization concentration of 0.001 wt %.

Comparative Experiment:

Effect of Farnesol on Sporulation of Aspergillus niger

TABLE 2

| | | | | | | |
|---|---|---|---|---|---|---|
| Farnesol concentration [μM] | 0 | 25 | 62.5 | 125 | 250 | 500 |
| Sporulation [%] | 100 | 90 | 75 | 50 | 10 | 0 |

### EXAMPLE 2

### Effect of Ortho-Phenylphenol/Silicic Acid Ester (OPP/SAE) on Sporulation of Aspergillus niger

The surfaces of wort agar plates were each contaminated with 100 μl of a germ suspension (10³ CFU/ml) of Aspergillus niger (DSM1988). Different quantities of active substance (solutions in ethanol, see table for final concentrations) had previously been added to the agar plates. The plates were incubated for 3 days at 25° C. Sporulation was assessed visually, and the sporulation rate (in %) was determined. The concentrations of active substance that were used did not prevent growth of the test strain.

TABLE 3
Ortho-phenylphenol/silicic acid ester

| | | | | | | | | |
|---|---|---|---|---|---|---|---|---|
| OPP/SEA concentration [%] | 0 | 0.00001 | 0.00005 | 0.0001 | 0.0005 | 0.001 | 0.005 | 0.01 |
| Sporulation [%] | 100 | 95 | 95 | 95 | 50 | 25 | 0 | 0 |

Sporulation was inhibited with increasing concentrations, and completely suppressed at a utilization concentration of 0.005 wt %.

### EXAMPLE 3

| Ingredients | Quantity |
|---|---|
| Methylhydroxyethyl cellulose (300 m · Pas in 2% aqueous | 500 g |
| solution, methoxyl content 26%) | |
| PVAcetate redispersion powder | 350 g |
| Kaolin | 60 g |
| Cellulose powder | 50 g |
| Addition product of 6 mol ethylene oxide and 1 mol | 10 g |
| nonylphenol | |
| Commercially available preservative (based on isothiazoline | 8 g |
| derivative) | |
| Ortho-phenylphenol | 0.1 g |

### EXAMPLE 4

| Ingredients | Quantity |
|---|---|
| Methylhydroxyethyl cellulose (5000 m · Pas in 2% aqueous | 680 g |
| solution, methoxyl content 19%) | |
| Carboxylmethyl starch (DS 0.22) | 300 g |
| Addition product of 4 mol ethylene oxide and 1 mol fatty | 15 g |
| alcohol | |
| Commercially available preservative (based on isothiazoline | 10 g |
| derivative) | |
| Ortho-phenylphenol | 0.1 g |

### EXAMPLE 5

| Ingredients | Quantity |
|---|---|
| Commercially available polyvinyl acetate dispersions (50% | 500 g |
| solids content) | |
| Water | 200 g |
| Methylhydroxyethyl cellulose (3000 m · Pas in 2% aqueous | 20 g |
| solution) | |
| Commercially available preservative | 10 g |
| Ortho-phenylphenol | 0.1 g |

The resulting mixtures were stirred with water at a ratio of 1:2 (2) or 1:25 (3) or 1:1 (4), and used to glue commercially available wallpapers onto wall surfaces.

### EXAMPLE 6

### Liquid Washing Agent

| | Quantity |
|---|---|
| Raw material | (wt %) |
| C₁₂-C₁₈ fatty alcohol + 7 EO (Dehydol LT 7, Cognis) | 15 |
| C₁₂-C₁₄ fatty alcohol + 2 EO sulfate, sodium salt | 7 |
| (Texapon N 70, Cognis) | |
| C₈₋₁₆ fatty acid cut (coconut oil fatty acid, Edenor | 8 |
| K12-18, Cognis) | |
| Sodium citrate | 1.5 |
| Enzymes | + |
| Dye | + |
| Perfume | + |
| Ortho-phenylphenol | 0.2 |
| Water | to make 100 |

What is claimed is:

1. A method of inhibiting asexual reproduction of fungi, comprising contacting a fungus with a composition comprising a non-fungicidal, non-fungistatic, asexual reproduction inhibitive concentration of o-phenylphenol or a derivative thereof.

2. The method of claim 1, wherein the o-phenylphenol or derivative thereof is bound in a carrier molecule.

3. The method of claim 1, wherein the carrier molecule comprises a clathrate molecule.

4. The method of claim 1, wherein the derivative of o-phenylphenol comprises an ester or ether thereof.

5. The method of claim 1, wherein the fungus is human-pathenogenic.

6. The method of claim 1, wherein the fungus is selected from the group consisting of the classes Ascomycota, Basidiomycota, Deuteromycota, and Zygomycota, the genera Aspergillus, Penicillium, Cladosporium, and Mucor, and all human-pathenogenic forms of Candida.

7. The method of claim 6, wherein the fungus is selected from the group consisting of Aspergillus aculeatus, Aspergillus albus, Aspergillus alliaceus, Aspergillus asperescens, Aspergillus awamori, Aspergillus candidus, Aspergillus carbonarius, Aspergillus carneus, Aspergillus chevalieri, Aspergillus chevalieri var. intermedius, Aspergillus clavatus, Aspergillus ficuum, Aspergillus flavipes, Aspergillus flavus, Aspergillus foetidus, Aspergillus fumigatus, Aspergillus giganteus, Aspergillus humicola, Aspergillus intermedius, Aspergillus japonicus, Aspergillus nidulans, Aspergillus niger, Aspergillus niveus, Aspergillus ochraceus, Aspergillus oryzae, Aspergillus ostianus, Aspergillus parasiticus, Aspergillus parasiticus var. globosus, Aspergillus penicilloides, Aspergillus phoenicis, Aspergillus rugulosus, Aspergillus sclerotiorum, Aspergillus sojae var. gymnosardae, Aspergillus sydowi, Aspergillus tamarii, Aspergillus terreus, Aspergillus terricola, Aspergillus toxicarius, Aspergillus unguis, Aspergillus ustus, Aspergillus versicolor, Aspergillus vitricolae and Aspergillus wentii.

8. The method of claim 1, wherein the composition further comprises at least one substance other than the o-phenylphenol or derivative thereof, said substance comprising a biocide present in the composition in a fungicidal concentration.

9. The method of claim 1, wherein the composition comprises 0.000001% to 2% by weight of ophenylphenol or a derivative thereof.

10. The method of claim 9, wherein the composition comprises 0.00001% to 1% by weight of o-phenylphenol or a derivative thereof.

11. The method of claim 10, wherein the composition comprises 0.00001% to 0.1% by weight of ophenylphenol or a derivative thereof.

12. The method of claim 11, wherein the composition comprises 0.00005% to 0,05% by weight of o-phenylphenol or a derivative thereof.

13. The method of claim 12, wherein the composition comprises 0.00005% to 0.0005% by weight of o-phenyiphenol or a derivative thereof.

14. The method of claim 10, wherein the composition comprises 0.001% to 1% by weight of o-phenylphenol or a derivative thereof.

15. The method of claim 14, wherein the composition comprises 0.001% to 0.1% by weight of o-phenylphenol or a derivative thereof.

16. The method of claim 1, wherein the fungus is present on a material selected from the group consisting of textiles, ceramics, metals, filter media, construction materials, construction adjuvants, furs, paper, hides, leather, and plastics.

17. A sealing compound comprising one or more acrylate or urethane polymers, one or more organic or inorganic fillers, and 0.000001% to 2% by weight of o-phenylphenol or a derivative thereof.

18. The sealing compound of claim 17, further comprising at least one other biocidal substance present in a fungicidal concentration.

19. An adhesive composition comprising at least one adhesive compound and 0.000001% to 2% by weight of o-phenylphenol or a derivative thereof.

20. The composition of claim 19, wherein the adhesive compound is water-soluble.
